(19) United States
(12) Patent Application Publication
(10) Pub. No.: US 2008/0317791 A1
(43) Pub. Date: Dec. 25, 2008

(54) Kappa Carrageenan

(75) Inventor: Jens Eskil Trudsoe, Roskilde (DK)

Correspondence Address: J M HUBER CORPORATION, 333 THORNALL STREET, PATENT DEPARTMENT, EDISON, NJ, 08837-2220, US

(73) Assignee: CP KELCO U.S., INC., Atlanta, GA (US)

(21) Appl. No.: 11/767,763

(22) Filed: Jun. 25, 2007

(51) Int. Cl. A23L 1/05; A23L 1/31; A61K 8/02; A61K 8/06; A61K 9/66; A61L 9/04

(52) U.S. Cl. 424/401; 424/456; 424/76.3; 426/574; 426/575

(57) ABSTRACT

Disclosed is a carrageenan composition comprising sodium, potassium, calcium and magnesium.

### BACKGROUND OF THE INVENTION

Production of carrageenan can be traced back to Ireland where plants of the red seaweed algae species of chrondrus crispus were first harvested with rakes during low tide or by gathering seaweed that had washed ashore. After harvesting, the weeds were typically washed, sun-bleached, dried and boiled with milk to form a pudding. The weeds themselves were dubbed “Irish Moss” and after making it familiar to most of Europe, Nineteenth Century Irish immigrants carried it to the U.S. and Canada as well.

Today, this seaweed pudding is mostly confined to Ireland's cultural history, but carrageenan has become much more important because of its effectiveness as a functional food additive in forming gels in an aqueous system, which make it useful in a wide variety of applications, including beer (in which it has been used for over 150 years as a fining) to processed meat and food products like milk drinks and deserts; pharmaceutical preparations such as orally-administered gelcaps; personal care products such as toothpaste and skin care preparations; and household products such air-freshener gel and cleaning gels. The temperature at which carrageenan gels and melts is dependent on a number of factors that include especially the concentration of gelling cations such as potassium and calcium ions. Generally speaking, the higher the concentration of gelling cations the higher the gelling and melting temperature of the carrageenan. Such cations may come not only from the composition to which the carrageenan is added as a gelling agent, but also from the carrageenan itself.

Thus, carrageenans with relatively high gelling cation concentrations also require relatively high-temperature processing. Generally, lower temperature processes are preferred since these save processing time, are less expensive and don't negatively affect the preparation of the composition in which the carrageenan is being included—this is especially important for food compositions, where higher temperatures may impair the base foodstuffs that are included in the food product. Thus, in order to produce carrageenan materials that promote gelling at even lower temperatures there is a continuing need for carrageenan extraction methods that reduce the concentration of gelling cations in the carrageenan.

Contemporary methods of carrageenan extraction and production have advanced considerably in the last fifty years. Perhaps most significantly is that today, rather than being gathered from wild-grown seaweed, carrageenan-containing plants such as Kappaphycus cottonii (Kappaphycus alvarezii), Euchema spinosum (Euchema denticulatum), and the above mentioned Chondrus crisus are more commonly seeded along nylon ropes and harvested in massive aqua-culture farming operations particularly in parts of the Mediterranean and throughout much of the Indian Ocean and along the Asian Pacific Ocean Coastline. Just as in the Nineteenth-century process, in contemporary processes before further processing the seaweed raw materials are first thoroughly cleaned in water to remove impurities and then dried. Then, as described in U.S. Pat. No. 3,094,517 to Stanley et al. the carrageenan is extracted from the cleaned seaweed while also at the same time being subjected to alkali modification by placing the seaweed in solution made slightly alkaline by the addition of a low concentration of alkali salt (i.e., a pH of the solution is raised to a range of, e.g., 9-10) and then heating this solution to a temperature of around 80° C. for a period of time of about 20 minutes to as long as two hours.

Subjecting the carrageenan-containing seaweed to alkali modification has the desired result of reducing the gelling cation concentration in the resulting carrageenan product; however, the extent to which the gelling cation levels can be reduced is limited because only relatively low concentrations of alkali may be used so as to not depolymerise (and thus damage) the carrageenan in the seaweed. So even though the gelling cation concentrations are reduced, they still remain high.

For example, when an alkali modification process is NOT used, typical cation concentration levels in kappa carrageenan are:
Potassium; About 5%
Calcium; About 0.4%
Magnesium; About 0.5%
Sodium: About 2%

When an alkali modification step is used to reduce these gelling cation concentrations, such as in U.S. Pat. No. 3,094,517 (Stanley et ah), which makes use of calcium hydroxide as alkali modification agent, the resulting cation concentration levels are:
Potassium; About 5%
Calcium; About 2%
Magnesium: About 0.01%
Sodium: About 1%

As can be seen, the alkali modification step taught in U.S. Pat. No. 3,094,517 significantly reduced the levels of magnesium and sodium ions, but not other gelling cations such as potassium and calcium.

Given the foregoing there is a need in the art for carrageenan materials containing lower concentrations of gelling cations.

### BRIEF SUMMARY OF THE INVENTION

Disclosed in the present invention is a carrageenan composition comprising: sodium in the range 4.720-6.960%, preferably 5.520-6.960% and most preferably 5.770-6.960%; potassium content of 0.015% -1.820%, preferably 0.015-0.036% and most preferably 0.015-0.025%; calcium content of 0.032-0.210%, preferably 0.032-0.134% and most preferably 0.032-0.077%; and magnesium content of 0.037-0.210%, preferably 0.037-0.086%, and most preferably 0.037-0.066%; wherein the carrageenan's gelling temperatures in the range 10-20° C., preferably 10-1.6° C. and most preferably 1.0-12° C.; and the carrageenan's melting temperatures in the range 22-36° C., preferably 22-29° C. and most preferably 22-23° C.

Disclosed in the present invention is a carrageenan composition comprising sodium in the range 4.390-5.730%, preferably 5.520-5.730% and most preferably 5.660-5.730%; potassium in the range of about 0.021-1.190%, preferably 0.021-0.090% and most preferably 0.021-0.024%; calcium in the range of 0.220-0.340%, preferably 0.220-0.270%, and most preferably 0.220-0.230%; and magnesium in the range of 0.041-0.170%, preferably about 0.041-0.061%, and most preferably 0.041-0.055%; wherein the carrageenan's gelling temperature is in the range 9-18° C.; and the carrageenan's melting temperatures in the range 21-34° C., preferably 21-26° C., and most preferably 21-24° C.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 shows the effect of treatment on gelling and melting temperatures in air freshener.

FIG. 2 shows the gelling and melting temperatures at 1% concentration of treated seaweed extracts in air freshener.

FIG. 3 shows break strength of treated seaweed extracts in air freshener.

FIG. 4 shows gel strength of treated seaweed extracts in air freshener.

FIG. 5 shows gelling and melting temperatures of treated seaweed extracts at different salt concentrations.

FIG. 6 shows gelling and melting temperatures of treated seaweed extracts at 3% sodium chloride.

FIG. 7 shows gelling and melting temperatures at different potassium chloride concentrations.

FIG. 8 shows the gel strength index in processed meat containing 2% salt and heated to a center temperature of 72° C.

FIG. 9 shows break strength index in processed meat containing 2% salt and heated to a center temperature of 72° C.

FIG. 10 shows the slope index of compression curve of processed meat containing 2% salt and heated to a center temperature of 72° C.

FIG. 11 shows syneresis index of processed meat containing 2% salt and heated to a center temperature of 72° C.

FIG. 12 shows gel strength index of processed meat containing 3% salt and heated to center temperatures of 68 and 72° C.

FIG. 13 shows break strength index of processed meat containing 3% salt and heated to center temperatures of 68 and 72° C.

FIG. 14 shows the slope of the compression curve index of processed meat containing 3% salt and heat treated at center temperatures of 68 and 72° C.

FIG. 15 shows the syneresis index of processed meat containing 3% salt and heat treated at center temperatures of 68 and 72° C.

FIG. 16 shows gel strength index in processed meat with 3% salt and heat treatment to center temperature 65° C.

FIG. 17 shows break strength index in processed meat with 3% salt and heat treatment to center temperature 65° C.

FIG. 18 shows the slope of compression curve index of processed meat with 3% salt and heat treatment to center temperature 65° C.

FIG. 19 shows syneresis index of processed meat with 3% salt and heat treatment to center temperature 65° C.

FIG. 20 shows gelling and melting temperatures with potassium concentration and diffusion time in an air freshener system.

FIG. 21 shows viscosity of toothpastes made with carrageenan product of the present invention and traditional carrageenan used in toothpaste.

FIG. 22 shows a temperature sweep graph.

FIG. 23 shows a temperature sweep graph

### DETAILED DESCRIPTION OF THE INVENTION

All parts, percentages and ratios used herein are expressed by weight unless otherwise specified. All documents cited herein are incorporated by reference.

By “alkali” it is meant a base according to the Brønsted-Lowry definition, i.e., an alkali is a molecule or ion that accepts a proton in a proton-transfer reaction.

The present invention is directed to kappa carrageenans, which may be more specifically described as generic repeating galactose and 3,6-anhydrogalactose residues linked b-(1-4) and a-(1-3), respectively and with characteristic 4-linked 3,6-anbydro-a-D-galactose and 3-linked-b-D-galactose-4-sulphate group—kappa carrageenans differ from iota carrageenans only by the presence of a single sulphate group. The molecules arrange themselves in a right-handed double helix with the strands parallel and threefold, again iota and kappa carrageenan are very similar in this regard, with kappa carrageenan forming a slightly more disordered helix. The helix is stabilized by interchain hydrogen bonds through the only unsubstituted positions at O-2 and O-6 with the sulphate groups projecting outward from the helix. As mentioned above, there is a strong correlation between the presence of gelling cations and gellation. Without being limited by theory, it is believed that gels are formed in kappa carrageenan through gelling (primarily monovalent) cations such as Na, K, Rb, Cs, NH₄, Ca²⁺ as well as some divalent cations like calcium atoms that facilitate side-by-side interaction of the strands to form a three dimensional gel network. The exact transformation mechanism from the carrageenan as randomly-oriented coils at higher temperatures to a gelled network is the subject of some dispute. As the temperature is lowered the random coils of carrageenan molecules reaggregate to form gels. In one model of gellation, a gel is created by the formation of the carrageenan molecules into double helices; in certain forms of carrageenan (such as kappa carrageenan) these double helices may themselves aggregate side-by-side due to the influence of the aforementioned gelling cations forming aggregates of double helices and eventually even footing domains of a three-dimensional ordered gel network. Alternatively it has been suggested that upon cooling the random coils of the carrageenan molecules do not form double helices but only single helix structures, and that these single helix structures form single helices in which the gelling cations nested in the bends of the helix promote Intermolecular aggregation.

Accordingly, the present invention is directed towards a process for treating fresh or dried kappa carrageenan-containing seaweed so as to substantially reduce to amount of gelling cations from the kappa carrageenan in the seaweed. Of equal importance is that this treatment process reduces the gelling cation concentration without extracting the carrageenan; in other words, depleting the gelling cations of the carrageenan by performing the alkali modification process essentially in situ. By modifying the polymer in situ in the seaweed, depolymerise of the carrageenan polymer is avoided and a kappa carrageenan preparation is produced that forms gels having lower gelling and melting temperatures than were hitherto known.

The process for producing kappa carrageenans according to the present invention will now be described in greater detail.

The present process utilizes a first step which is a conventional cleaning step in which the carrageenan-containing seaweed, particularly seaweed of the species Euchema cottonii, is washed to remove impurities and unwanted particulates. The water may be sea water, tap water, rain water, deionised water, sodium chloride softened water or preferably demineralised water. Washing may be conducted at temperatures in the range 5-25° C. The washing may be conducted as a counter current wash or a batch wash, with a counter current process preferred because of its better utilisation of the treatment liquid. (In all subsequent steps of the process of the present invention, the water may be rain water, deionised water, sodium chloride softened water, but preferably demineralised water).

The second step in the process may be practiced in accordance with three different embodiments.

### (a) Second Step, First Embodiment

In the first embodiment, the second step is a treatment of the cleaned seaweed with an aqueous treatment solution containing alkali in water. The alkali provides cations, which exclude potassium, calcium and/or magnesium in the carrageenan, while the concentration of the alkali in the treatment solution is held sufficiently high to reduce the aqueous solubility of the carrageenan thus preventing it from leaching out of the seaweed and dissolving into the water during this and subsequent steps.

Accordingly, by treating the carrageenan-containing seaweed in this way, the carrageenan is depleted from its gelling cat ions in situ.

Preferred alkalis are sodium hydroxide and its corresponding carbonates and bicarbonates, with sodium hydroxide being the most preferred. Sodium hydroxide is particularly notable for reducing the gelling and melting temperatures of carrageenan. Also suitable is calcium hydroxide As discussed above, the concentration of the alkali must be such to provide sufficient cations while preventing solubilization of the carrageen in the water phase; an appropriate range to accomplish this dual purpose is a concentration of alkali in range of 3-30 wt %, preferably 10-25 wt % and most preferably 15-20 wt %.

In some cases alcohol may be added to the treatment solution to further reduce the leaching out of the carrageenan from the seaweed and its dissolving into water. It is particularly important to add alcohol when relatively small quantities of the aqueous treatment liquid are used. This is because excess water initially present in the wet seaweed and also remaining from the washing step could dilute the concentration of the cations in the aqueous treatment solution to the point that the carrageenan begins to leach out. The presence of alcohol in the treatment solution helps maintain high yields, especially as the treatment temperature is increased. Preferred alcohols are methanol, ethanol and isopropyl alcohol with ethanol being most preferred. The amount of alcohol ranges from 200-800 ml alcohol per 1000 ml treatment solution, preferably 200-600 ml alcohol per 1000 ml treatment solution and most preferably 500-600 ml alcohol per 1000 ml treatment solution.

The temperature during treatment ranges from 0-70° C., preferably 5-50° C. and most preferably 5-35° C. The treatment time is in the range 10 minutes-24 hours, preferably 10 minutes-4 hours, and most preferably 15 minutes-80 minutes. Either a batch wise or counter current process may be used; although as mentioned above the counter current process is preferred because it makes better utilisation of the treatment liquid.

Carrageenan products according to the first embodiment produce gels having gelling temperatures in the range 10-20° C., preferably 10-16° C. and most preferably 10-12° C.; and melting temperatures in the range 22-36° C., preferably 22-29° C. and most preferably 22-23° C. In addition, carrageenan products according to the first embodiment are characterized by a sodium content in the range 4.720-6.960%, preferably 5.520-6.960% and most preferably 5.770-6.960%; a potassium content of 0.015% -1.820%, preferably 0.015-0.036% and most preferably 0.015-0.025%; a calcium content of 0.032-0.210%, preferably 0.032-0.134% and most preferably 0.032-0.077%; and a magnesium content of 0.037-0.210%, preferably 0.037-0.086%, and most preferably 0.037-0.066%.

### (b) Second Step, Second Embodiment

In a second embodiment of the present invention, the second step is a treatment of the washed seaweed with an aqueous treatment solution containing a salt. The effect is similar as described above with respect to the first embodiment where the salt provides monovalent cations to prevent the diffusion of potassium, calcium and magnesium ions into the carrageenan while the concentration of the sodium salt in the treatment solution is held sufficiently high to reduce the aqueous solubility of the carrageenan thus reducing its leaching out from seaweed and dissolution into water. Thus similarly as above, by treating the carrageenan-containing seaweed in this way, the carrageenan is depleted from its gelling cat ions in situ.

Salts include sodium salts like sodium chloride, sodium sulphate, sodium phosphate, sodium tripolyphosphate and sodium hexametaphosphate. The concentration of sodium salt in the water phase is in the range 3-30 wt %, preferably 10-25 wt %, and more preferably 15-20 wt %,

As described above in the section entitled “Second Step, First Embodiment”, alcohol may optionally be added to the treatment solution to further reduce the leaching out of the carrageenan from the seaweed and dissolving into water. Similarly, the same temperature and time parameters are used in this embodiment of the process as in the previous two mentioned above.

In this embodiment, the temperature during treatment ranges from 0-25° C., preferably 0-10° C., and more preferably 0-5° C. The treatment time is in the range 10 minutes-24 hours, preferably 10 minutes-4 hours, and most preferably 10 minutes-40 minutes. Either a batch wise or counter current process may be used; the counter current process is preferred because it makes better utilisation of the treatment liquid.

Carrageenan products according to the second embodiment produce gels having gelling temperatures in the range 9-18° C.; and melting temperatures in the range 21-34° C., preferably 21-26° C. and most preferably 21-24° C. In addition, carrageenan products according to the second embodiment are characterized by a sodium content in the range 4.390-5.730%, preferably 5.520-5.730% and most preferably 5.660-5.730%; a potassium content of 0.021-1.190%, preferably 0.021-0.090% and most preferably 0.021-0.024%; a calcium content of 0.220-0.340%, preferably 0.220-0.270% and most preferable 0.220-0.230.%; and a magnesium content of 0.041-0.170%, preferable 0.041-0.061% and most preferably 0.041-0.055%.

### (C) Second Step, Third Embodiment

In a third embodiment of the present invention, this second step is essentially split into two substeps which include a first substep of treating the washed seaweed with a first aqueous treatment solution containing about 3-30 wt %, preferably 10-25 wt %, and most preferably 15-20 wt %, of an alkali, then a second substep of treating the alkali-treated seaweed with a second aqueous treatment solution containing about 3-30 wt %, preferably 10-25 wt %, and most preferably 15-20 wt %, of a salt. (For purposes of clarity, exactness and completeness to persons of ordinary skill in the art these substeps are referred to as separate processing steps in the claims.) Suitable salt and alkali species are set forth above.

As described above in the section entitled “Second Step, First Embodiment”, alcohol may optionally be added to the treatment solution to further reduce the leaching out of the carrageenan from the seaweed and dissolving into water. Similarly, the same temperature and time parameters are used in this embodiment of the process as in the previous two mentioned above.

Carrageenan products according to the third embodiment of the second step produce gels having gelling temperatures in the range 9-19° C., preferably 9-15° C. and most preferably 9-13° C., and melting temperatures in the range 21-35° C., preferably 21-29° C. and most preferably 21-26° C. In addition, carrageenan products according to the third embodiment of the second step are characterized by a sodium content in the range 4.870-6.910%, preferably 5.770-6.910% and more preferably 6.010-6.910%; a potassium content of 0.014-1.180%, preferably 0.014-0.068% and more preferably 0.014-0.035%; a calcium content of 0.073-0.260%, preferably 0.073-0.200% and most preferably 0.073-0.146%; and a magnesium content of 0.010-0.290%, preferably 0.010-0.1.60% and more preferably 0.010-0.103%.

### (D) Second Step, Fourth Embodiment

In a third embodiment of the present invention, the second step is treating the washed seaweed with an aqueous treatment solution containing both an alkali and a salt. The solution contains about 3-15 wt %, preferably 5-15 wt %, most preferably 5-10 wt %, of an alkali, and about 3-15 wt %, preferably 5-15 wt %, and most preferably 5-10 wt % of a salt. Suitable salt and alkali species are set forth above.

As described above in the section entitled “Second Step, First Embodiment”, alcohol may optionally be added to the treatment solution to further reduce the leaching out of the carrageenan from the seaweed and dissolving into water. Similarly, the same temperature and time parameters are used in this embodiment of the process as in the previous two mentioned above.

The temperature during treatment ranges from 0-70° C. preferably 5-50° C. and most preferably 5-35° C. The treatment time is in the range 10 minutes-24 hours, preferably 10 minutes-4 hours, and most preferably 15 minutes-80 minutes. Either a batch wise or counter current process may be used; the counter current process is preferred because it makes better utilisation of the treatment liquid.

Carrageenan products according to the fourth embodiment produce gels having gelling temperatures in the range 9-19° C., preferably 9-15° C. and most preferably 9-13° C.; and melting temperatures in the range 21-35° C., preferably 21-29° C. and most preferably 21-26° C. In addition, carrageenan products according to the fourth embodiment of the second step are characterized by a sodium content in the range 4.870-6.910%, preferably 5.770-6.910% and more preferably 6.010-6.910%, a potassium content of 0.014-1.180%, preferably 0.014-0.068% and more preferably 0.014-0.035%; a calcium content of 0.073-0.260%, preferably 0.073-0.200% and most preferably 0.073-0.146%; and a magnesium content of 0.010-0.290%, preferably 0.010-0.160% and more preferably 0.010-0.103%.

In the third step in the process (which is common to all three embodiments of the second step discussed above) the treated seaweed is subjected to washing to remove the excess of the last reagent that was used in the second or treatment step. The reagent can of course be either an salt or an alkali. Washing is done with slow agitation and the number of washings is in the range 1-4, preferably 1-2, and washing time is in the range 10-30 minutes per wash, preferably 15 minutes per wash. Controlling the number of washing steps is important because the yield decreases with time (possible reasons for this are discussed below) and because the number of washing steps affects the gelling and melting temperatures (again, this is discussed in greater detail, below). As above to limit leaching out of the carrageenan from the seaweed the temperature during washing is held in the range 0-25° C., preferably 0-5° C.

In the fourth and final step of the process the treated seaweed can be dried and ground into a powder of semi-refined carrageenan products, which in addition to carrageenan also contain the cellulosic material from the seaweed.

Alternatively, pure carrageenan can be extracted from the treated seaweed in pure water, such as one of the water types described above (again demineralised water is preferred). Of primary importance is that the extraction step does not re-introduce the gelling cations. Extraction temperatures are in the range 0-90° C., preferably 25-90° C. and most preferably 50-90° C. Typically, higher extraction temperatures result in greater yields.

Other aspects of the processes for production of carrageenan according to the present invention are not particularly limited, and where necessary conventional carrageenan technology may be used. In addition to the specific steps set forth herein, processes of the present invention may further comprise additional processes typically associated with carrageenan production.

An additional important aspect of this present invention is that because the relationship between the gelling and melting temperatures and the several processing parameters has been determined with such specificity, then these temperatures can be controlled depending on the specific properties desired in the carrageenan. In other words, by specially controlling the processing parameters, a carrageenan having particular properties can be produced.

The present invention will now be explained in greater details with respect to the following several experiments. These experiments and their accompanying textual descriptions, will present detailed descriptions of the process of the present invention as well as results obtained from the experimental process. Additionally analysis of the results will be presented and supplemented by possible theoretical explanations. The following experimental equipment, materials and methods were used in carrying out the present experiments. Application of these experimental methods are introduced in the specific examples section below that illustrate the present invention and place it within the context of the prior art.

Equipment
Hobart mixer equipped with heating and cooling jacket and stirrer—Hobart N-50G produced by Hobart Corporation, USA.
Cooling unit capable of cooling to about 5° C., e.g., the Haake K10/Haake DC 10 produced by Thermo Electron GmbH, Germany.
Magnetic stirrer and heater equipped with temperature control, e.g., Ikamag Ret produced by Janke & Kunkel GmbH, Germany.
Beakers, 1 litre and 2 liters.
2 liters conical flask, Büchner funnel and vacuum pump.
Filter cloth.
Rheoraeter—Haake RheoStress RS100 equipped with cup Z20/48 mm and rotor Z20 DIN produced by Thermo Electron GmbH, Germany.
pH-meter—PHM220 produced by Radiometer, Denmark
Analytical balance, weighing with two decimals—Sartorius Basic B3100P produced by Sartorius GmbH, Germany.
Texture analyzer TA-TX2
Crystallizing dishes having diameter of about 50 mm and height of about 20 mm.

Chemicals:
Sodium chloride, analytical, Merck KGaA, Darmstadt, Germany
Calcium chloride dehydrate, analytical, Merck, Germany
Sodium hydroxide, analytical, Merck, Germany
Potassium hydroxide, analytical, Merck
Calcium hydroxide, analytical, Merck
Sodium sulphate, analytical, Merck
Sodium methyl-4-hydroxybenzoate, analytical, Merck
Potassium chloride, analytical, Merck
Tri sodium phosphate dodecahydrate, analytical, Merck
Ethanol, 96%
Methanol, 100%
Isopropyl alcohol, 100%
Potassium chloride, analytical, Merck
Glycerine, analytical, Scharlau Chemie, Barcelona, Spain
Lemon oil, H. N. Fusgaard, Roedovre, Denmark
Cremophor RH 40, BASF, Ludwigshafen, Germany

Treatment of seaweed:
1. Seaweed was washed three times in 1 litre demineralized water and refrigerated.
2. This washed seaweed was then placed in a 2-litre beaker.
3. A treatment solution was formed by the salt or alkali was dissolved at room temperature in 1000 ml of demineralized water, and subsequently cooled to the treatment temperature.
4. Seaweed was added to the treatment solution.
5. Seaweed was treated at specific temperatures and times (see below) while being stirred.
6. Treated seaweed was washed in demineralized water at specific temperatures and times (see below).
7. The washed seaweed was extracted in 1500 ml. demineralised water at 90° C. for 1 hour.
8. The extract was filtered on diatomaceous earth.
9. The filtered extract was precipitated in three volumes 100% IPA and the precipitate was washed in 1 litre 100% IPA.
10. The washed precipitate was dried at 70° C. overnight.
11. The dry precipitate was milled on 0.25 mm screen.

The Determination of gelling and melting temperatures of carrageenan-compositions was made using a composition with the following carrageen-incorporating composition:

| Ingredients | Grams | % |
|---|---|---|
| Seaweed extract | 0.48 | 0.96 |
| Glycerine | 3.00 | 6.00 |
| Parabene | 0.05 | 0.10 |
| Demineralized | 33.75 | 67.50 |
| Water | | |
| Lemon oil | 1.25 | 2.50 |
| Isopropyl alcohol | 1.50 | 3.00 |
| Cremophor RH 40 | 10.00 | 20.00 |
| Net weight | 50.00 | 100.00 |

This composition was prepared as follows:
1. The water, glycerine and parabene were mixed.
2. The seaweed extract was dispersed in this mixture and stirred for about 60 minutes.
3. The dispersion was heated while stirring to 70° C.
4. The dispersion was then cooled to 55-60° C.
5. A hot (about 50° C.) preparation of oil, isopropyl alcohol and Cremophor RH 40 was mixed into the cooled dispersion.
6. The net weight was adjusted with hot (about 60° C.) water and cooled over night at room temperature.

The gelling and melting temperatures were measured by temperature sweeps on Haake RheoStress RS100, using cooling and heating rates of 1° C./min. The following program was generally used, however, in some instances where gelling and melting temperatures were higher; the program was run at higher starting temperatures and lower end-temperatures:
1. 65-5° C., 0,50 Pa, f=0,4640 Hz
2. 5-65° C., 0,50 Pa, f=0,4640 Hz
3. Gelling temperature is defined as the temperature during the cooling sweep, where the elastic modulus, G′ intersects with the viscous modulus, G″.
4. Melting temperature is defined as the temperature during the heating sweep, where the elastic modulus, G′ intersects with the viscous modulus, G″.

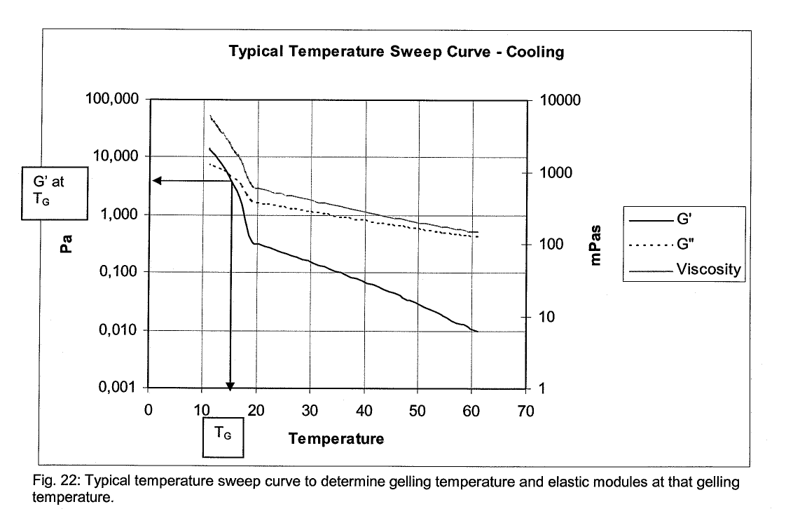

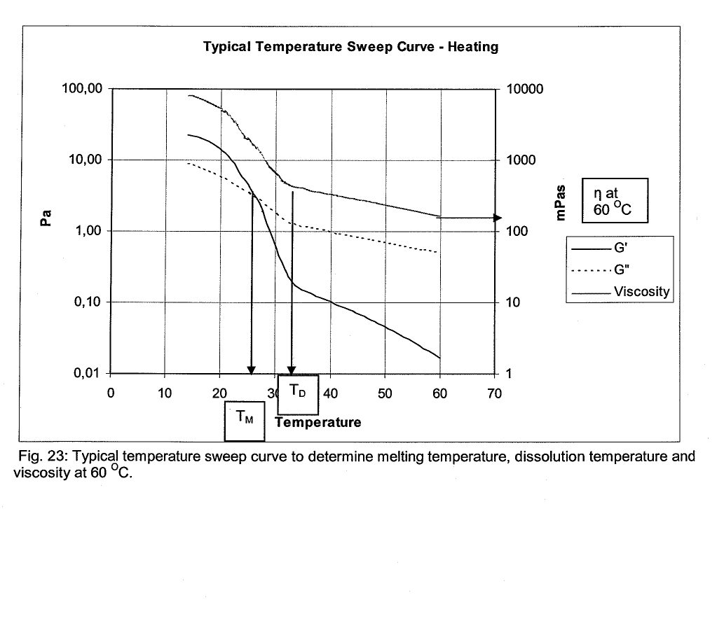

FIG. 22 and FIG. 23 show typical temperature sweep graphs. The determination of break strength and gel strength of carrageenan-compositions was made using a composition with the following carrageen-incorporating composition:

| Ingredients | Grams | % |
|---|---|---|
| Seaweed extract | 0.96 | 0.96 |
| Glycerine | 12.00 | 6.00 |
| Parabene | 0.20 | 0.10 |
| Demineralized | 134.80 | 67.40 |
| Water | | |
| Potassium chloride | 0.40 | 0.20 |
| Lemon oil | 5.00 | 2.50 |
| Isopropyl alcohol | 6.00 | 3.00 |
| Cremophor RH 40 | 40.00 | 20.00 |
| Net weight | 200.00 | 100.00 |

This composition was prepared as follows:
1. The water, glycerine, parabene and potassium chloride were mixed.
2. The seaweed extract was dispersed in this mixture and stirred for about 60 minutes.
3. The dispersion was heated while stirring to 70° C.
4. The dispersion was then cooled to 55-60° C.
5. A hot (about 50° C.) preparation of oil, isopropyl alcohol and Cremophor RH 40 was mixed into the cooled dispersion.
6. The net weight was adjusted with hot (about 60° C.) water.
7. The hot solution was poured into crystallizing dishes having adhesive tape round to brim making it possible to fill the crystallizing dish to above the brim and cooled over night at room temperature.

The break strength and gel strength were measured on Texture analyzer TA-TX2. Break strength is the load needed to break the gel and gel strength is the load needed to deform the gel by 2 mm.

The following procedure was used for gelling and melting temperatures in demineralized water:
1. The carrageenan product was added slowly at room temperature to demineralized water while stirring on magnetic stirrer. Stirring was continued until the preparation was completely lump-free.
2. The preparation was then heated while stirring on magnetic stirrer to 70° C., and left to cool at room temperature.

The following procedure for gelling and melting temperatures in demineralized water with salts:
1. The salt was dissolved in demineralized water at room temperature.
2. The carrageenan product was added slowly to the salt solution at room temperature while stirring on magnetic stirrer.
3. The preparation was then heated while stirring on magnetic stirrer to up to 90° C., and left to cool at room temperature.
   The Viscosity in Toothpaste was measured using the following equipment, chemicals, formula, and procedure:

Equipment
1. Beaker, 100, 1
2. Beaker, 150 ml, height 95 mm, diameter 50 mm
3. Analytical balance
4. Laboratory scale, max load: 7000 g, precision: 0, 1 g
5. Electric stirrer, Janke and Kunkel GmbH type RW20
6. Household mixer, Hobart type N-50
7. Brookfield viscosimeter RVT
8. Brookfield Helipath Stand D
9. Low temperature incubator, 25° C.
10. High temperature incubator, 50° C.
11. Thermostatically controlled water bath at 25° C., Haake F3-K
12. Nesco film
13. Stopwatch
14. Plastic lids

Chemicals
Glycerol, 100%
Dicalcium phosphate dehydrate, CaHPO4, 2H2O
Tetra sodium pyrophosphate decahydrate, Na4O7P2, 10 H2O, Sieved through a 40 mesh
Sodium chloride, NaCl

Formula

| | | |
|---|---|---|
| Carrageenan product | 6.60 | g |
| Glycerol | 220.00 | g |
| Dicalcium phosphate dehydrate | 480.00 | g |
| Tetra sodium pyrophosphate decahydrate | 4.20 | g |
| Sodium chloride | 6.70 | g |
| Deionized water | 282.50 | g |
| Total | 1000.00 | g |

Process
1. Carrageenan product was dispersed in glycerol in exactly 3 minutes while stirring with a propeller stirrer (200-400 rpm), which was stirred “for another 10 minutes (400 rpm).
2. Additional water was added while stirring (800 rpm). And the speed increased to 1200 rpm after 5 minutes and then mixed for another 10 minutes.
3. The solution was transferred to the household mixer quantitatively.
4. The tetra sodium pyrophosphate was added during mixing (speed 1) and stirred for 5 minutes (speed 2).
5. The dicalcium phosphate dehydrate was added at speed 1 and mixed for 15 minutes (speed 2). The bowl and blade was scraped after 1, 5 and 10 minutes respectively.
6. The sodium chloride was added and mixed for 25 minutes (speed 2). The bowl and blade was scraped after 5, 10 and 15 minutes respectively while maintaining a smooth texture to the paste.
7. The paste was placed into four 150 ml beakers and covered with plastic lids making sure that as little air as possible is introduced in the paste during filling.
8. The 4 beakers were placed in a water bath—which was pre-adjusted to 25° C.—for 1 hour—while making sure that all of the paste in the beakers was below the water level.
9. The toothpastes were covered tightly with Nesco-film.
10. Two beakers were then placed in the low-temperature incubator (adjusted to 25° C.) and two beakers were placed in a high-temperature incubator (adjusted to 50° C.).
11. After 3 days' storage, one beaker was transferred from the high-temperature incubator to a 25° C water bath and kept there for 1 hour. Viscosity was measured 72 hours after the start of the incubation.
12. There was then a measurement of the two 3-days viscosities at 25 ° C. (after storage at 25° C. and 50° C., respectively) on Brookfield Viscosimeter RVT with Helipath Stand, 2.5 rpm by using the following spindles:
   Toothpaste stored at 25° C.: Spindle T-D
   Toothpaste stored at 50° C. Spindle T-E
13. Both the pointer and the zero-point were placed in the middle of the window on the Brookfield and the spindle placed just below the surface. The Brookfield and Helipath stand were started just after the spindle has run 3 times.
14. Three readings were taken for each measurement, and the relative Brookfield units were the average readings multiplied by the following spindle factors:
   Factor Spindle T-D=8
   Factor Spindle T-E=20
15. After 7 days' storage, the second beaker was transferred from the high-temperature incubator to a 25° C. water bath and kept there for 1 hour.
16. The two 7-days viscosities were measured at 25° C. (after storage at 25° C. and 50° C., respectively) and the relative Brookfield units were calculated as described in step 12.

The following procedure was used for determination of Break Strength and Gel Strength of Cold set Air Freshener Gel:

| Ingredients | Grams | % |
|---|---|---|
| Seaweed extract | 0.58 | 0.97 |
| Glycerine | 3.60 | 6.00 |
| Parabene | 0.06 | 0.10 |
| Water | 40.44 | 67.40 |
| Lemon oil | 1.50 | 2.50 |
| Isopropyl alcohol | 1.80 | 3.00 |
| Mulsifan RT-141 | 12.00 | 20.00 |
| Net weight | 60.00 | 99.97 |

Procedure:
1. Water, glycerine and parabene was mixed at room temperature while stirring until dissolved.
2. Carrageenan was added slowly at room temperature and mixing was continued for about 60 minutes until carrageenan was fully dissolved without any lumps.
3. Lemon oil, isopropanol and emulsifier were mixed at room temperature to full dissolution of oil and alcohol.
4. The oil phase was added to the water phase at room temperature while stirring to ensure complete mixing of the two phases.
5. Dry potassium chloride was added to the bottom of a crystallizing dish.
6. The mixture of the water phase and the oil phase was poured over the dry potassium chloride at room temperature and left at room temperature to gel.
7. Measure break strength and gel strength over a period of 72 hours.

The invention will now be described in more detail with respect to the following non-limiting examples which were performed with the above described equipment, materials and methods.

### EXAMPLES

T_{G} and T_{M} stand for gelling temperature and melting temperature, respectively, while T_{D} is the dissolution temperature, and η stands for intrinsic viscosity at 60° C. The “% yield” is calculated as: % yield=(g. dry precipitate×1500×100)/(g. seaweed×g. precipitated extract×seaweed dry matter). Since yield of polymer from seaweed changes with season and with seaweed harvesting location, the yield of neutral extractions of seaweed have been assigned an index of 100, and subsequent calculations of yield index utilize that baseline figure.

Several products were prepared from carrageenan compositions prepared according to the present invention using Eucheuma cottonii seaweed. These carrageenan compositions where made using one of the following process methods: (1) Combined Alkali and Salt Treatment; (2) Salt treatment; and (3) Alkali treatment.

Combined Alkali and Salt Treatment. First, the washed seaweed was treated with 170 g sodium hydroxide dissolved in 700 ml demineralized water and 300 ml ethanol at 5° C. The treated seaweed was then washed once for 15 minutes in a mixture of 800 ml demineralized water and 200 ml ethanol at25° C. However, in some runs, the ethanol concentration was increased. Afterwards, the alkali treated seaweed was treated at 25° C. with 200 g sodium chloride dissolved in 1000 ml demineralized water. Lastly, the salt treated seaweed was washed twice for 15 minutes with a mixture of 800 ml demineralized water and 200 ml ethanol at 25° C. However, in some runs the ethanol concentration was increased. For comparison, a number of neutral extractions were performed. The results are listed in Tables 1-3, below.

TABLE 1

| | | | | | | | | G′ | | | | | |
|---|---|---|---|---|---|---|---|---|---|---|---|---|---|
| | | | | | | | | At | | | | | |
| | | Amount | | | T_{G} | T_{M} | T_{D} | T_{G} | η | Na | K | Ca | Mg |
| Sample | Seaweed g | Precipitated g | Precipitate g | Yield % | ° C. | ° C. | ° C. | Pa | cP | Mg/g | Mg/g | Mg/g | Mg/g |
| Neutral | 41.18 | 606.26 | 3.47 | 77.42 | 32 | 54 | 57 | 2.2 | 200 | 17.30 | 48.40 | 3.80 | 4.30 |
| Neutral | 38.54 | 714.42 | 3.85 | 77.88 | 31 | 52 | 54 | 2.5 | 210 | 16.50 | 47.20 | 3.70 | 4.80 |
| Neutral | 33.61 | 626.56 | 2.97 | 78.56 | 32 | 51 | 53 | 2.2 | 200 | 18.10 | 44.50 | 4.20 | 4.30 |
| Neutral | 33.72 | 691.36 | 3.28 | 78.37 | 32 | 52 | 54 | 2.2 | 180 | 16.80 | 45.00 | 4.10 | 4.60 |
| Neutral | 36.73 | 659.56 | 3.30 | 75.87 | 32 | 52 | 54 | 2.5 | 180 | 17.20 | 46.40 | 4.00 | 4.60 |
| Average | 36.76 | 659.63 | 3.37 | 77.62 | 31.8 | 52.2 | 54.4 | 2.3 | 194 | 17.18 | 46.30 | 3.96 | 4.52 |

TABLE 2

| | | | EtOH | | | | | |
|---|---|---|---|---|---|---|---|---|
| | Alkali | EtOH | In | | | | | |
| | Treatment | In first | Second | | | | | |
| | Time | Wash | Wash | | Amount | | | Yield |
| Sample | Minutes | ml/1000 ml | ml/1000 ml | Seaweed g | Precipitated g | Precipitate g | Yield % | Index |
| Alkali | 90 | 200 | 200 | 37.57 | 627.96 | 2.69 | 63.51 | 82 |
| and salt | | | | | | | | |
| Alkali | 90 | 200 | 200 | 37.44 | 552.51 | 2.44 | 65.70 | 85 |
| and salt | | | | | | | | |
| Alkali | 90 | 200 | 200 | 33.13 | 665.24 | 2.64 | 66.72 | 86 |
| and salt | | | | | | | | |
| Average | | | | | | | 65.31 | 84 |
| Alkali | 90 | 200 | 500 | 35.35 | 507.27 | 2.34 | 72.68 | 94 |
| and salt | | | | | | | | |
| Alkali | 90 | 500 | 600 | 32.11 | 600.80 | 2.40 | 69.29 | 89 |
| and salt | | | | | | | | |
| Alkali | 90 | 500 | 500 | 31.16 | 505.33 | 2.05 | 72.52 | 93 |
| and salt | | | | | | | | |
| Average | | | | | | | 71.50 | 92 |

TABLE 3

| | Alkali | | | | | | | | | |
|---|---|---|---|---|---|---|---|---|---|---|
| | Treatment | | | | G′ At | | | | | |
| | Time | T_{G} | T_{M} | T_{D} | T_{G} | η | Na | K | Ca | Mg |
| Sample | Minutes | ° C. | ° C. | ° C. | Pa | cP | Mg/g | Mg/g | Mg/g | Mg/g |
| Alkali | 90 | 11 | 22 | 30 | 6.0 | 280 | 54.60 | 0.44 | 2.10 | 0.78 |
| and salt | | | | | | | | | | |
| Alkali | 90 | 9 | 21 | 29 | 6.1 | 210 | 55.90 | 0.29 | 2.30 | 0.68 |
| and salt | | | | | | | | | | |
| Alkali | 90 | 10 | 22 | 30 | 6.5 | 250 | 55.20 | 0.68 | 2.10 | 0.85 |
| and salt | | | | | | | | | | |
| Average | | 10 | 21.7 | 29.7 | 6.2 | 247 | 55.23 | 0.47 | 2.17 | 0.77 |
| Alkali | 90 | 10 | 21 | 29 | 5.2 | 180 | 57.10 | 0.25 | 2.20 | 0.84 |
| and salt | | | | | | | | | | |
| Alkali | 90 | 10 | 24 | 31 | 6.0 | 280 | 55.30 | 0.61 | 2.00 | 0.80 |
| and salt | | | | | | | | | | |
| Alkali | 90 | 10 | 21 | 30 | 6.5 | 190 | 55.10 | 0.24 | 1.90 | 0.74 |
| and salt | | | | | | | | | | |
| Average | | 10.0 | 22.0 | 30.0 | 5.9 | 217 | 55.83 | 0.37 | 2.03 | 0.79 |

Salt treatment. The washed seaweed was treated with 140 g sodium chloride dissolved in 700 ml demineralized water and 300 ml ethanol at 5° C. After treatment, the treated seaweed was washed twice for 15 minutes with a mixture of 500 ml demineralized water and 500 ml ethanol at 25° C.

TABLE 4

| | Treatment | | | | | | | | | G′ At | | | | | |
|---|---|---|---|---|---|---|---|---|---|---|---|---|---|---|---|
| | Time | | Amount | | | Yield | T_{G} | T_{M} | T_{D} | T_{G} | η | Na | K | Ca | Mg |
| Sample | Minutes | Seaweed g | Precipitated g | Precipitate g | Yield % | Index | ° C. | ° C. | ° C. | Pa | cP | Mg/g | Mg/g | Mg/g | Mg/g |
| Neutral | | 29.59 | 705.38 | 2.96 | 90.37 | 100 | 31 | 53 | 56 | 3.1 | 290 | 19.50 | 38.40 | 3.80 | 4.50 |
| Salting | 10 | 35.37 | 543.77 | 2.65 | 87.80 | 97 | 18 | 34 | 37 | 6.0 | 500 | 46.70 | 10.20 | 3.00 | 1.60 |
| Salting | 10 | 32.14 | 536.14 | 2.19 | 80.99 | 90 | 14 | 31 | 34 | 6.0 | 300 | 50.00 | 7.60 | 3.40 | 1.23 |
| Salting | 10 | 30.82 | 626.48 | 2.46 | 81.19 | 90 | 17 | 34 | 37 | 5.0 | 380 | 43.90 | 11.90 | 3.40 | 1.70 |
| Salting | 10 | 30.50 | 652.74 | 2.47 | 79.06 | 87 | 16 | 32 | 35 | 6.0 | 390 | 45.40 | 10.40 | 3.20 | 1.60 |
| Salting | 10 | 31.25 | 565.30 | 2.30 | 82.96 | 92 | 18 | 34 | 37 | 6.0 | 400 | 45.70 | 9.60 | 3.20 | 1.40 |
| Average | | | | | 82.40 | 91 | 16.6 | 33.0 | 36.0 | 5.8 | 394 | 46.34 | 9.94 | 3.24 | 1.51 |
| Salting | | | | | | | | | | | | | | | |
| Salting | 60 | 31.91 | 553.67 | 2.43 | 87.64 | 97 | 12 | 25 | 31 | 6.8 | 330 | 52.70 | 2.30 | 2.70 | 0.76 |
| Salting | 60 | 32.40 | 516.79 | 2.25 | 85.63 | 95 | 10 | 23 | 29 | 7.0 | 300 | 54.50 | 1.50 | 2.70 | 0.61 |
| Salting | 60 | 31.04 | 665.68 | 2.78 | 85.73 | 95 | 12 | 26 | 31 | 6.0 | 410 | 52.60 | 2.20 | 2.80 | 0.80 |
| Salting | 60 | 32.18 | 616.68 | 2.41 | 77.38 | 86 | 9 | 21 | 28 | 5.9 | 310 | 54.20 | 1.20 | 2.70 | 0.65 |
| Salting | 60 | 31.87 | 592.48 | 2.33 | 78.63 | 87 | 11 | 24 | 31 | 5.9 | 250 | 53.80 | 1.50 | 2.70 | 0.62 |
| Average | | | | | 83.00 | 92 | 10.8 | 23.8 | 30.0 | 6.3 | 320 | 53.56 | 1.74 | 2.72 | 0.69 |
| Salting | | | | | | | | | | | | | | | |

Alkali Treatment. In this example, several runs were made of alkali treatment, alone. The washed seaweed was treated with 140 g sodium hydroxide dissolved in 700 ml demineralized water and 300 ml ethanol at 5° C. After treatment, the treated seaweed was washed twice for 15 minutes in a mixture of 500 ml demineralized water and 500 ml ethanol at 25° C.

TABLE 5

| | Treatment | | | | | | | | | G′ At | | | | | |
|---|---|---|---|---|---|---|---|---|---|---|---|---|---|---|---|
| | Time | | Amount | | | Yield | TG | TM | TD | TG | η | Na | K | Ca | Mg |
| Sample | Minutes | Seaweed g | Precipitated g | Precipitate g | Yield % | Index | OC | OC | OC | Pa | cP | Mg/g | Mg/g | Mg/g | Mg/g |
| Neutral | | 30.76 | 644.42 | 2.81 | 92.81 | 100 | 32 | 52 | 55 | 4.0 | 310 | 18.50 | 41.20 | 3.90 | 4.40 |
| Alkali | 10 | 32.39 | 659.92 | 2.42 | 74.13 | 80 | 18 | 35 | 37 | 5.0 | 360 | 47.90 | 16.10 | 0.94 | 0.46 |
| Alkali | 10 | 35.57 | 553.28 | 2.32 | 77.18 | 83 | 20 | 36 | 39 | 6.0 | 450 | 47.20 | 18.20 | 0.92 | 0.41 |
| Alkali | 10 | 33.03 | 637.74 | 2.33 | 72.42 | 78 | 18 | 34 | 38 | 6.0 | 480 | 49.10 | 13.00 | 1.00 | 0.50 |
| Alkali | 10 | 35.79 | 598.44 | 2.43 | 74.28 | 80 | 19 | 35 | 38 | 6.0 | 430 | 48.90 | 13.20 | 1.10 | 0.56 |
| Average | | | | | 74.50 | 80 | 18.8 | 35.0 | 38.0 | 5.8 | 430 | 48.28 | 15.13 | 0.99 | 0.48 |
| Alkali | | | | | | | | | | | | | | | |
| Alkali | 90 | 35.25 | 566.24 | 2.34 | 76.76 | 83 | 13 | 28 | 33 | 6.5 | 230 | 57.90 | 3.40 | 0.63 | 0.46 |
| Alkali | 90 | 33.52 | 664.42 | 2.32 | 68.20 | 73 | 13 | 29 | 33 | 7.0 | 290 | 57.60 | 3.70 | 0.85 | 0.48 |
| Alkali | 90 | 33.85 | 685.50 | 2.50 | 70.54 | 76 | 14 | 29 | 33 | 6.0 | 190 | 60.00 | 4.30 | 0.50 | 0.38 |
| Alkali | 90 | 34.43 | 612.12 | 2.46 | 76.42 | 82 | 14 | 29 | 33 | 6.2 | 310 | 56.70 | 5.70 | 0.65 | 0.40 |
| Alkali | 90 | 36.05 | 604.72 | 2.58 | 77.49 | 83 | 14 | 30 | 33 | 6.0 | 250 | 57.30 | 5.30 | 0.48 | 0.39 |
| Average | | | | | 73.88 | 80 | 13.6 | 29.0 | 33.0 | 6.3 | 254 | 57.90 | 4.48 | 0.62 | 0.42 |
| Alkali | | | | | | | | | | | | | | | |

Cold Air Freshener Composition. The carrageenan materials produced according to the above procedures outlined in Tables 1-5 were then incorporated into a cold air freshener composition (the composition is set forth above, along with the process used to make it). The cold air freshener compositions were then tested and the results are set forth in Table 6, below.

TABLE 6

| | | | | G′ At | | | | | |
|---|---|---|---|---|---|---|---|---|---|
| | | T_{G} | T_{M} | T_{G} | T_{D} | 0.2% KCl | | | |
| Sample | Carrageenan % | ° C. | ° C. | Pa | ° C. | Carrageenan % | BS g | GS g | Comment |
| Neutral | 0.50 | 24 | 45 | 1.0 | 47 | 0.50 | 23.7 | 14.3 | Dry gel |
| | 1.00 | 31 | 52 | 2.0 | 57 | 1.00 | 101.6 | 49.9 | Dry gel |
| | 1.50 | 36 | 58 | 3.0 | 60 | 1.50 | 235.9 | 102.3 | Dry gel |
| Alkali and | 0.50 | 11 | 13 | 1.0 | 29 | 0.50 | 24.0 | 14.0 | Dry gel |
| Salt | 1.00 | 10 | 23 | 1.4 | 30 | 0.75 | 53.9 | 25.1 | Dry gel |
| | 1.50 | 13 | 28 | 9.0 | 31 | 1.00 | 87.3 | 43.5 | Dry gel |
| Salt 10 | 0.50 | 10 | 31 | 1.1 | 35 | 0.50 | 25.5 | 12.9 | Dry gel |
| | 1.00 | 16 | 33 | 5.0 | 36 | 1.00 | 101.1 | 43.4 | Dry gel |
| | 1.50 | 20 | 37 | 10.0 | 39 | 1.33 | 160.4 | 65.8 | Dry gel |
| Salt 60 | 0.50 | 3 | 13 | 1.7 | 28 | 0.50 | 23.5 | 12.3 | Dry gel |
| | 1.00 | 10 | 26 | 6.0 | 31 | 1.00 | 80.9 | 39.6 | Dry gel |
| | 1.50 | 13 | 28 | 10.1 | 31 | 1.33 | 128.2 | 66.3 | Dry gel |
| Alkali 10 | 0.50 | 11 | 31 | 2.3 | 36 | 0.50 | 26.7 | 15.8 | Dry gel |
| | 1.00 | 19 | 35 | 6.0 | 37 | 1.00 | 108.9 | 47.0 | Dry gel |
| | 1.50 | 22 | 38 | 10.0 | 41 | 1.33 | 166.1 | 78.1 | Dry gel |
| Alkali 90 | 0.50 | 6 | 21 | 2.2 | 31 | 0.50 | 28.0 | 15.3 | Dry gel |
| | 1.00 | 14 | 30 | 5.0 | 33 | 1.00 | 105.3 | 48.7 | Dry gel |
| | 1.50 | 17 | 33 | 9.0 | 35 | 1.33 | 179.3 | 79.7 | Dry gel |
| Traditional | 0.50 | 32 | 53 | 0.7 | 56 | 0.50 | 159.4 | 72.0 | Wet gel |
| Kappa | 1.00 | 38 | 63 | 1.8 | 66 | 1.00 | 574.2 | 294.4 | Wet gel |
| | 1.50 | 44 | 71 | 2.0 | 73 | 1.33 | 943.8 | 428.7 | Wet gel |

The samples in Table 6 are set forth according to their method of manufacturing. Thus, the neutral extracted were combined into one sample, which is termed “Neutral”. The samples made through alkali treatment for 90 minutes and salt treatment for 60 minutes were combined and termed “Alkali and Salt”. The samples made through treatment with salt for 10 minutes were combined and termed “Salt 10”. The samples treated with salt for 60 minutes were combined and termed “Salt 60”. The samples treated with alkali for 10 minutes were combined and termed “Alkali 10”. And the samples treated with alkali for 90 minutes were combined and termed “Alkali 90”. For comparison, test were also run using “Traditional Kappa”, which is a kappa carrageenan extracted from Eucheuma cottonii according to the teaching of U.S. Pat. No. 3,094,517 in combination with U.S. Pat. No. 3,907.770. )

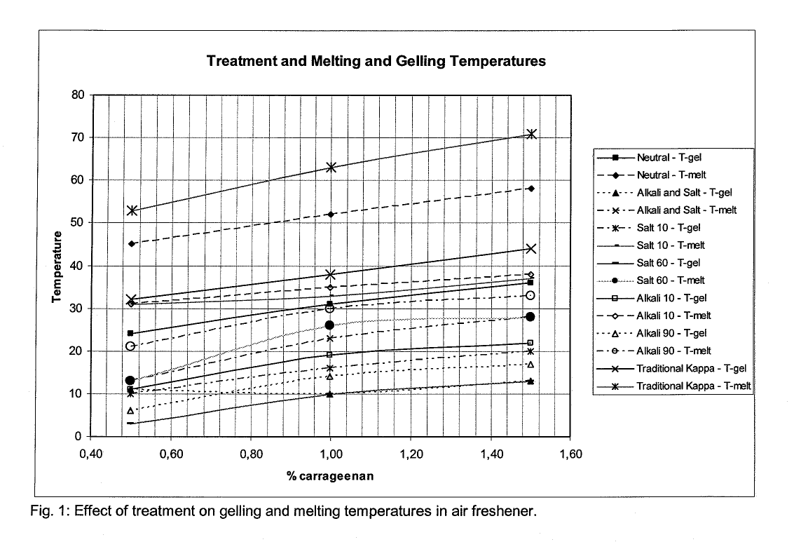

FIG. 1 shows that “Traditional Kappa” has the highest melting and gelling temperatures. Neutral also produced relatively high temperatures. The lowest melting and gelling temperatures are made with NaCl for 60 minutes, then treatment with NaOH for 90 minutes. Interestingly, the treatment involving both NaOH and NaCl provides gelling temperatures which are relatively constant over the concentration range 0.5-1.5%. Next to follow is treatment with NaCl for 10 minutes and lastly treatment with NaOH for 10 minutes

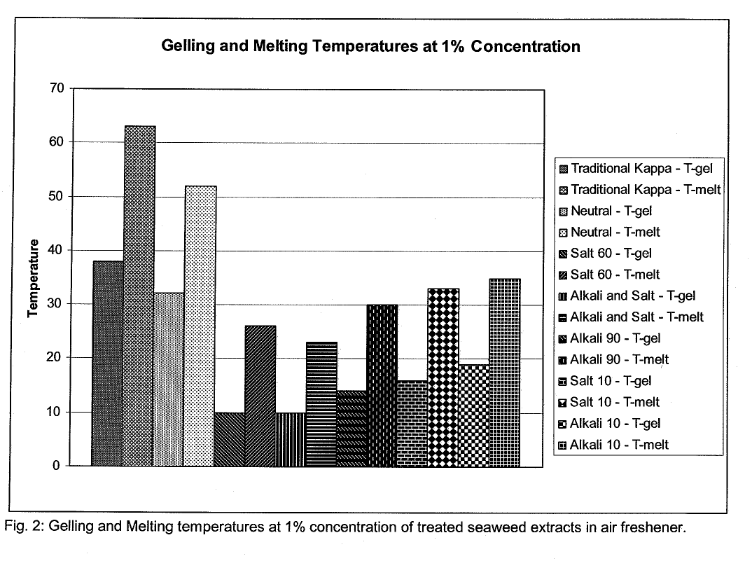

FIG. 2 shows that at 1% concentrations, the lowest gelling temperature and melting temperatures are achieved using a treatment with NaCl alone or with a combination of NaOH and NaCl. The latter is most likely to also dissolve at room temperature. With these treatments, melting can be achieved in the range 23° C.-63° C., and one might make a three layer gel, with each part melting at 23, at 35 and at 52° C.

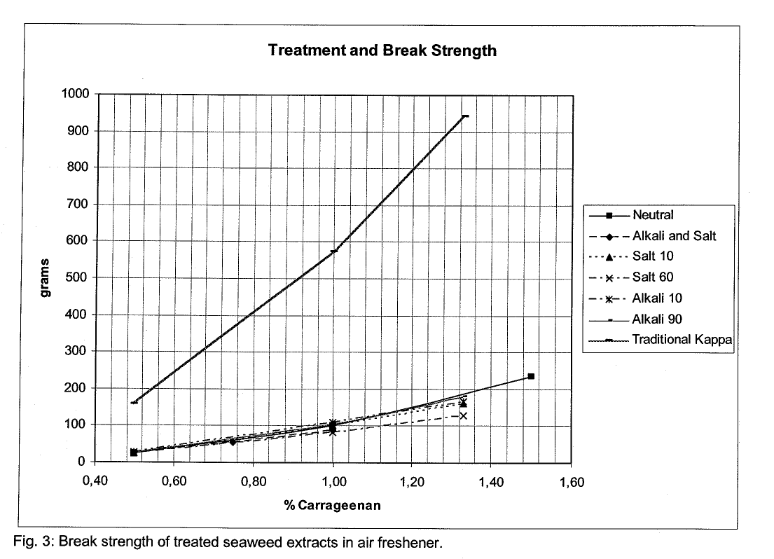

From FIG. 3, it is evident that treatments according to the present invention provide for substantially lower break strengths than break strengths of traditional kappa carrageenan. Lowest break strengths are achieved with a treatment for 60 minutes with NaCl and with the combined treatment with NaOH and NaCl. Note, however, that all gels with traditional kappa carrageenan provided wet gels.

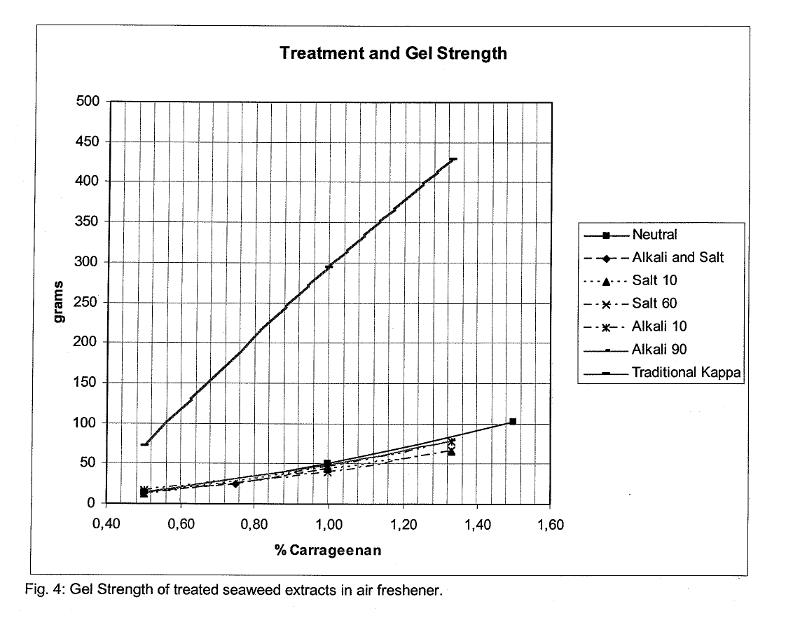

FIG. 4 shows that again, traditional kappa carrageenan provides substantially higher gel strength—about six fold. Little difference was observed among the various treatments according to the present invention including non-treatment. The treatments involving NaCl alone or a combination of NaOH and NaCl produced the lowest gel strengths.

Salt solution Compositions. The carrageenan material produced according to the above procedures outlined in Tables 1-5 were incorporated into a salt solution, specifically sodium chloride in demineralized water. Salt solutions of different concentrations were then tested and the results are set forth in Table 7, below.

TABLE 7

| | | | | | G′ At | |
|---|---|---|---|---|---|---|
| | | | T_{G} | T_{M} | T_{G} | T_{D} |
| Sample | Carrageenan % | NaCl % | ° C. | ° C. | Pa | ° C. |
| Neutral | 0.60 | 1.00 | 21 | 36 | 0.28 | 38 |
| | 0.60 | 3.00 | 25 | 35 | 0.19 | 38 |
| | 0.60 | 5.00 | 29 | 34 | 0.45 | 38 |
| Alkali and | 0.60 | 1.00 | 4 | 9 | 0.80 | 18 |
| Salt | 0.60 | 3.00 | 20 | 27 | 0.60 | 29 |
| | 0.60 | 5.00 | 31 | 37 | 0.40 | 46 |
| Salt 10 | 0.60 | 1.00 | 2 | 10 | 0.60 | 25 |
| | 0.60 | 3.00 | 24 | 28 | 0.40 | 31 |
| | 0.60 | 5.00 | 32 | 37 | 0.33 | 46 |
| Salt 60 | 0.60 | 1.00 | 4 | 9 | 0.50 | 22 |
| | 0.60 | 3.00 | 19 | 25 | 0.50 | 27 |
| | 0.60 | 5.00 | 29 | 34 | 0.27 | 37 |
| Alkali 10 | 0.60 | 1.00 | 9 | 19 | 0.60 | 27 |
| | 0.60 | 3.00 | 22 | 26 | 0.50 | 28 |
| | 0.60 | 5.00 | 35 | 39 | 0.40 | 41 |
| Alkali 90 | 0.60 | 1.00 | 3 | 9 | 0.70 | 22 |
| | 0.60 | 3.00 | 18 | 24 | 0.60 | 26 |
| | 0.60 | 5.00 | 33 | 39 | 0.60 | 41 |
| Traditional | 0.60 | 1.00 | 25 | 48 | 1.00 | 52 |
| Kappa | 0.60 | 3.00 | 38 | 63 | 0.52 | 68 |
| | 0.60 | 5.00 | 56 | 90 | 0.18 | 95 |

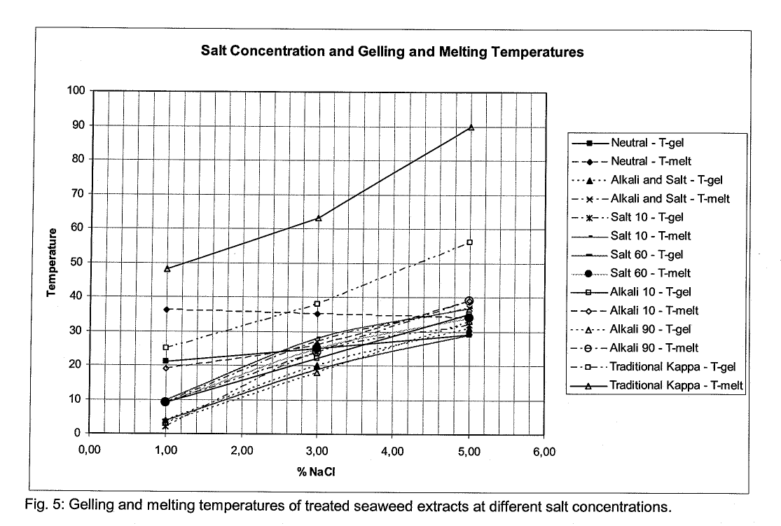

FIG. 5 shows that traditional kappa carrageenan provides for gelling and melting temperatures substantially higher than kappa carrageenan made according to the present invention. At 3% salt, traditional carrageenan displays melting temperature above 60° C.

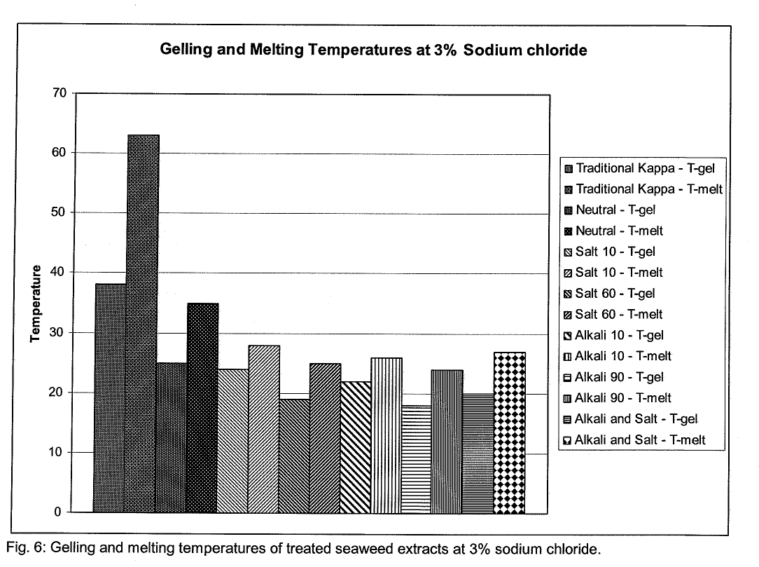

FIG. 6 shows that at a sodium chloride concentration of 3% in demineralized water, all treated seaweed extracts of the present invention display gelling and melting temperatures substantially below those of traditional kappa carrageenan. In fact, all gels made with treated seaweed extract of the present invention would melt at body temperature, both in the mouth and on the skin.

Salt solutions: potassium chloride. The carrageenan material produced according to the above procedures outlined in Tables 1-5 were incorporated into solutions of potassium chloride in demineralized water. Solutions of different concentrations were then tested and the results are set forth in Table 8, below.

TABLE 8

| | | | | | G′ At | | | |
|---|---|---|---|---|---|---|---|---|
| | | | T_{G} | T_{M} | T_{G} | T_{D} | | |
| Sample | Carrageenan % | KCl % | ° C. | ° C. | Pa | ° C. | BS g | GS g |
| Neutral | 1.00 | 0.00 | 9 | 28 | 2.00 | 32 | No | No |
| | | | | | | | gel | gel |
| | 1.00 | 0.05 | 21 | 41 | 1.00 | 42 | 9 | 7 |
| | 1.00 | 0.20 | 34 | 55 | 0.28 | 57 | 27 | 20 |
| | 1.00 | 0.30 | 41 | 62 | 0.40 | 67 | 37 | 28 |
| Alkali and | 1.00 | 0.00 | <5 | <5 | — | <5 | No | No |
| Salt | | | | | | | gel | gel |
| | 1.00 | 0.05 | 7 | 18 | 1.80 | 24 | No | No |
| | | | | | | | gel | gel |
| | 1.00 | 0.20 | 27 | 48 | 0.70 | 51 | 20 | 12 |
| | 1.00 | 0.30 | 36 | 53 | 0.20 | 55 | 36 | 21 |
| Salt 10 | 1.00 | 0.00 | <5 | <5 | — | <5 | No | No |
| | | | | | | | gel | gel |
| | 1.00 | 0.05 | 9 | 27 | 2.80 | 30 | No | No |
| | | | | | | | gel | gel |
| | 1.00 | 0.20 | 29 | 46 | 1.00 | 48 | 25 | 16 |
| | 1.00 | 0.30 | 39 | 60 | 0.60 | 63 | 47 | 26 |
| Salt 60 | 1.00 | 0.00 | <5 | <5 | — | <5 | No | No |
| | | | | | | | gel | gel |
| | 1.00 | 0.05 | 3 | 13 | 1.50 | 22 | No | No |
| | | | | | | | gel | gel |
| | 1.00 | 0.20 | 27 | 49 | 0.70 | 51 | 16 | 11 |
| | 1.00 | 0.30 | 37 | 54 | 0.45 | 57 | 30 | 23 |
| Alkali 10 | 1.00 | 0.00 | <5 | 7 | — | 10 | No | No |
| | | | | | | | gel | gel |
| | 1.00 | 0.05 | 10 | 21 | 0.60 | 25 | No | No |
| | | | | | | | gel | gel |
| | 1.00 | 0.20 | 29 | 45 | 0.70 | 48 | 22 | 12 |
| | 1.00 | 0.30 | 43 | 53 | 0.50 | 55 | 41 | 23 |
| Alkali 90 | 1.00 | 0.00 | <5 | <5 | — | <5 | No | No |
| | | | | | | | gel | gel |
| | 1.00 | 0.05 | 8 | 23 | 2.00 | 27 | No | No |
| | | | | | | | gel | gel |
| | 1.00 | 0.20 | 27 | 46 | 0.60 | 48 | 21 | 11 |
| | 1.00 | 0.30 | 40 | 51 | 0.20 | 54 | 38 | 23 |
| Traditional | 1.00 | 0.00 | 22 | 46 | 0.50 | 48 | 36 | 25 |
| Kappa | 1.00 | 0.05 | 28 | 49 | 0.20 | 52 | 228 | 83 |
| | 1.00 | 0.20 | 40 | 55 | 0.12 | 58 | 397 | 126 |
| | 1.00 | 0.30 | 42 | 64 | 2.00 | 68 | 484 | 132 |

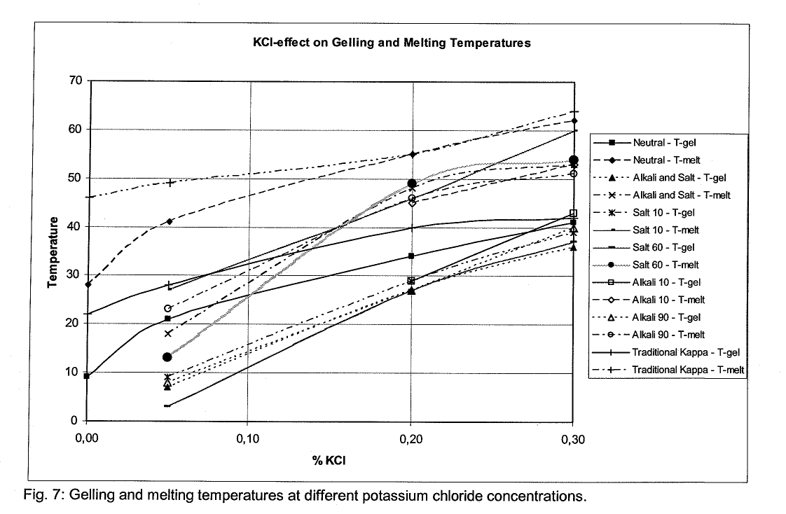

FIG. 7 shows that the gelling and melting temperatures increase approximately linearly with increasing KCl concentration. Highest temperatures are provided by traditional extract material, but pretty closely followed by neutral extraction. The treated extracts are all substantially lower and in general they are more steep, thus, more dependant on the KCl concentration. Accordingly, KCl can be used to further control gelling and melting temperatures.

Demineralized water. The carrageenan material produced according to the above procedures outlined in Tables 1-5 were incorporated into solutions of potassium chloride in demineralized water then tested with the results being set forth in Table 9, below.

TABLE 9

| | | T_{G} | T_{M} | G′ At T_{G} | T_{D} |
|---|---|---|---|---|---|
| Sample | Carrageenan % | ° C. | ° C. | Pa | ° C. |
| Neutral | 1.0 | 10 | 28 | 1.3 | 33 |
| Neutral | 2.0 | 24 | 45 | 1.5 | 48 |
| Neutral | 3.0 | 28 | 52 | 3.3 | 54 |
| Alkali 10 | 1.0 | <5 | <5 | — | <5 |
| Alkali 10 | 2.0 | 10 | 18 | 10.6 | 23 |
| Alkali 10 | 3.0 | 15 | 29 | 12.3 | 32 |
| Alkali | 1.0 | <5 | <5 | — | <5 |
| and Salt | | | | | |
| Alkali | 2.0 | <5 | <5 | — | <5 |
| and Salt | | | | | |
| Alkali | 3.0 | <5 | <5 | — | <5 |
| and Salt | | | | | |

Lowest temperatures are achieved with the combined treatment with alkali and salt. Also, compared to neutral extract, the treated products provide for higher G′.

Use in processed meat. The sample “Alkali and Salt” were tested in processed. For comparison, processed meat was made with no carrageenan, with “Traditional Kappa” and with a semi refined carrageenan made according to U.S. Pat. No. 5,801,240 and termed SRC. The results are set forth in Tables 10-12, below.

TABLE 10

| | | | Center | | | | | | | | | | | | |
|---|---|---|---|---|---|---|---|---|---|---|---|---|---|---|---|
| | | Salt | Temperature | | GS | | BS | Slope | Slope | | Total | | | Synerese | Synerese |
| Carrageenan | % | % | ° C. | GS g | Index | BS g | Index | g/mm | Index | Casing g | g | Drained g | Water g | % | Index |
| None | 0.00 | 2.00 | 72 | 2850 | 100 | 3595 | 100 | 415 | 100 | 14 | 882 | 783 | 85 | 9.8 | 100 |
| Alkali and | 0.25 | 2.00 | 72 | 2994 | 105 | 4906 | 136 | 439 | 106 | 14 | 880 | 805 | 61 | 7.0 | 72 |
| Salt | | | | | | | | | | | | | | | |
| Alkali and | 0.50 | 2.00 | 72 | 3838 | 135 | 6341 | 176 | 576 | 139 | 14 | 870 | 804 | 52 | 6.1 | 62 |
| Salt | | | | | | | | | | | | | | | |
| Alkali and | 0.75 | 2.00 | 72 | 4432 | 156 | 8080 | 225 | 659 | 159 | 14 | 866 | 800 | 52 | 6.1 | 62 |
| Salt | | | | | | | | | | | | | | | |
| Traditional | 0.50 | 2.00 | 72 | 3803 | 133 | 6653 | 185 | 566 | 136 | 14 | 900 | 833 | 53 | 6.0 | 61 |
| Kappa | | | | | | | | | | | | | | | |
| SRC | 0.50 | 2.00 | 72 | 3876 | 136 | 5321 | 148 | 556 | 134 | 14 | 866 | 784 | 68 | 6.8 | 69 |

TABLE 11

| | | | Center | | | | | | | | | | | | |
|---|---|---|---|---|---|---|---|---|---|---|---|---|---|---|---|
| | | | Temperature | | GS | | BS | Slope | Slope | Casing | Total | | | Synerese | Synerese |
| Carrageenan | % | Salt % | ° C. | GS g | Index | BS g | Index | g/mm | Index | g | g | Drained g | Water g | % | Index |
| None | 0.00 | 3.00 | 68 | 2487 | 100 | 4163 | 100 | 378 | 100 | 14 | 832 | 748 | 70 | 8.6 | 100 |
| SRC | 0.50 | 3.00 | 68 | 2836 | 114 | 4364 | 105 | 369 | 98 | 14 | 870 | 795 | 61 | 7.1 | 83 |
| Traditional | 0.50 | 3.00 | 68 | 3035 | 122 | 4477 | 108 | 453 | 120 | 14 | 815 | 746 | 55 | 6.9 | 80 |
| Kappa | | | | | | | | | | | | | | | |
| Alkali and | 0.50 | 3.00 | 68 | 3285 | 132 | 5455 | 131 | 480 | 127 | 14 | 850 | 788 | 48 | 5.7 | 67 |
| Salt | | | | | | | | | | | | | | | |
| None | 0.00 | 3.00 | 72 | 2514 | 100 | 3434 | 100 | 373 | 100 | 14 | 833 | 761 | 58 | 7.1 | 100 |
| SRC | 0.50 | 3.00 | 72 | 3136 | 125 | 5026 | 146 | 448 | 120 | 14 | 874 | 812 | 48 | 5.6 | 79 |
| Traditional | 0.50 | 3.00 | 72 | 3187 | 127 | 4828 | 141 | 432 | 116 | 14 | 822 | 756 | 52 | 6.4 | 91 |
| Kappa | | | | | | | | | | | | | | | |
| Alkali and | 0.50 | 3.00 | 72 | 3414 | 136 | 5555 | 162 | 470 | 126 | 14 | 887 | 822 | 51 | 5.8 | 82 |
| Salt | | | | | | | | | | | | | | | |

TABLE 12

| | | | Center | | | | | | | | | | | | |
|---|---|---|---|---|---|---|---|---|---|---|---|---|---|---|---|
| | | | Temperature | | GS | | BS | Slope | Slope | Casing | Total | | | Synerese | Synerese |
| Carrageenan | % | Salt % | ° C. | GS g | Index | BS g | Index | g/mm | Index | g | g | Drained g | Water g | % | Index |
| None | 0.00 | 3.00 | 65 | 421 | 100 | 709 | 100 | 65 | 100 | 14 | 860 | 737 | 109 | 12.9 | 100 |
| SRC | 0.50 | 3.00 | 65 | 259 | 62 | 558 | 79 | 45 | 69 | 14 | 871 | 781 | 76 | 8.9 | 69 |
| Traditional | 0.50 | 3.00 | 65 | 348 | 83 | 745 | 105 | 60 | 92 | 14 | 855 | 763 | 78 | 9.3 | 72 |
| Kappa | | | | | | | | | | | | | | | |
| Alkali and | 0.50 | 3.00 | 65 | 372 | 88 | 804 | 113 | 61 | 94 | 14 | 834 | 759 | 61 | 7.4 | 58 |
| Salt | | | | | | | | | | | | | | | |

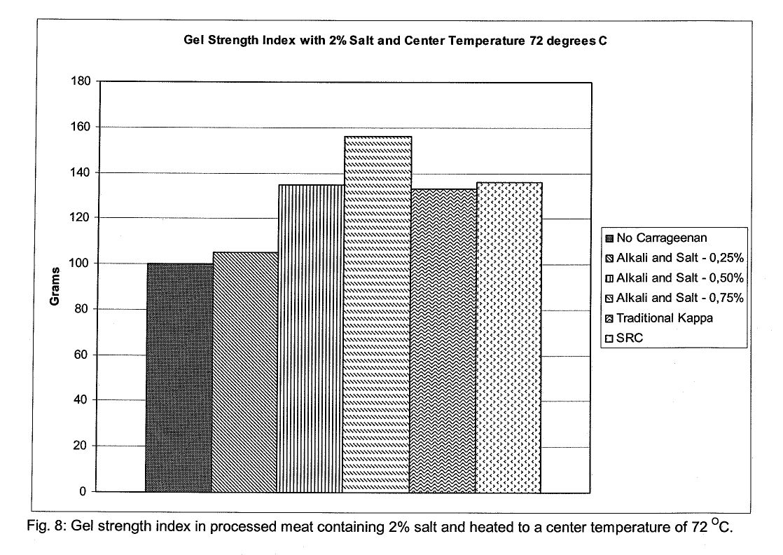

FIG. 8 shows that at 0.50% seaweed extract concentration, there is no difference in gel strength between traditional kappa carrageenan and semi refined carrageenan and the extracts of the present invention when processed meat contains 2% salt and is heated to a center temperature of 72° C.

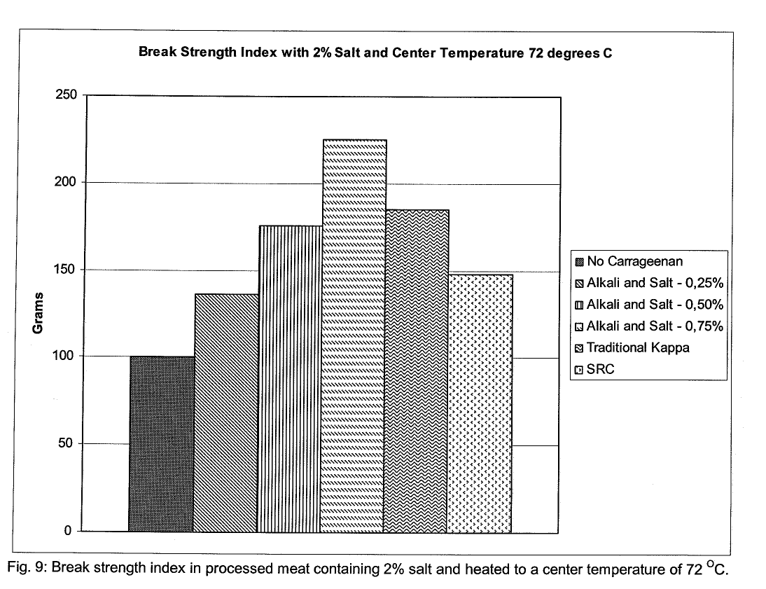

Likewise, FIG. 9 shows that at 0,50% seaweed extract concentration, there is no difference in break strength between traditional carrageenan and extracts of the present invention when processed meat contains 2% salt and is heated to a center temperature of 72° C. However, semi refined carrageenan at a concentration of 0,50% provides about the same break strength as the extract of the present invention in 0,25% concentration.

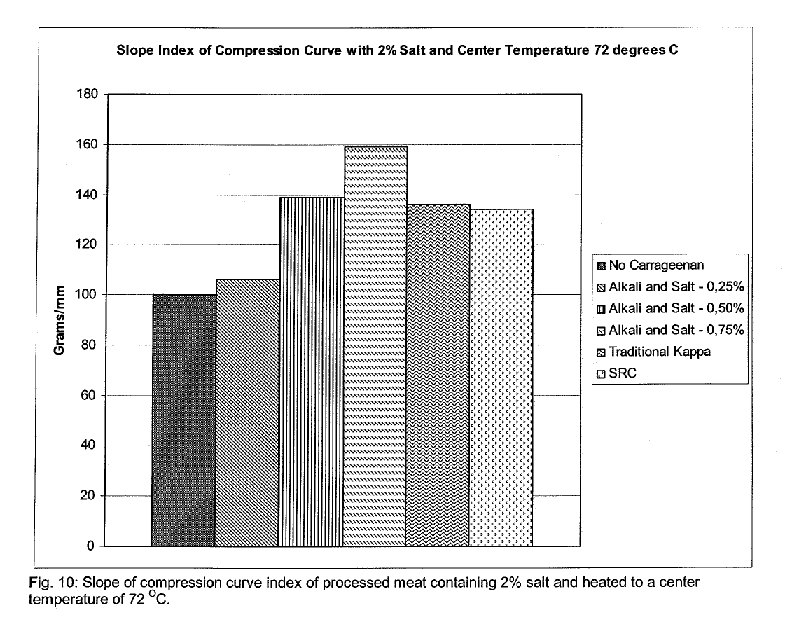

Similarly, FIG. 10 shows that at 0,50% seaweed extract concentration, there is no difference in the slope of the compression curve between traditional carrageenan and semi refined carrageenan and the extract of the present invention when processed meat contains 2% salt and is heated to a center temperature of 72° C.

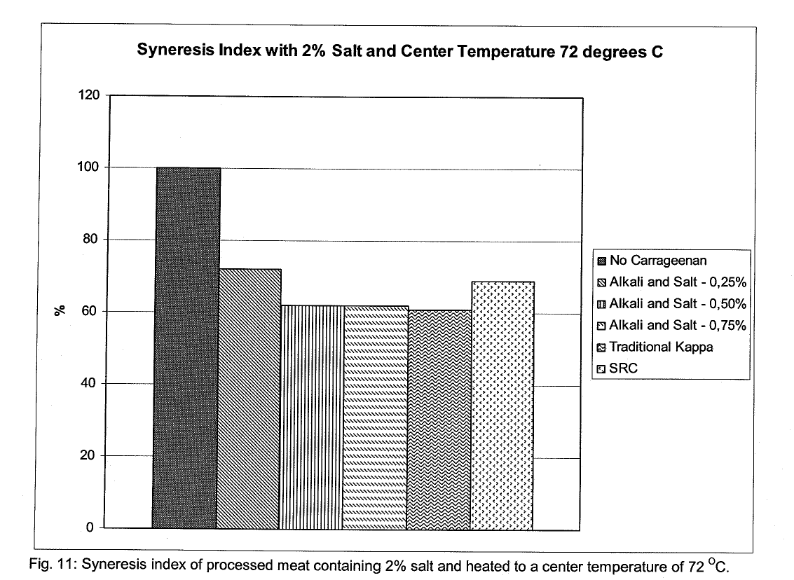

With a salt concentration of 2% and a center temperature of 72° C., FIG. 11 shows that syneresis of traditional kappa carrageenan, semi refined carrageenan and extracts of the present invention are the same. However, syneresis of 0,50% semi refined carrageenan displays about the same syneresis as the extract of the present inventing in 0,25% concentration.

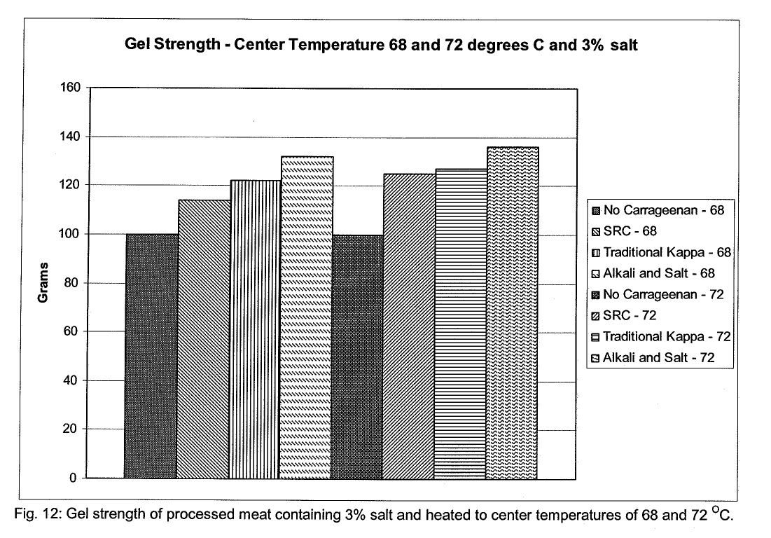

At 3% sait, FIG. 12 shows that the extract of the present invention provides processed meat, which is higher in gel strength at center temperatures of 72 and 68° C. Without being bound of theory, the extracts of the present invention are more soluble than traditional kappa carrageenan and semi refined carrageenan. Thus, as dissolution is impaired through higher salt concentration and/or lower processing times, the extracts of the present invention still maintain sufficient solubility to be able to form a strong and cohesive gel in processed meat.

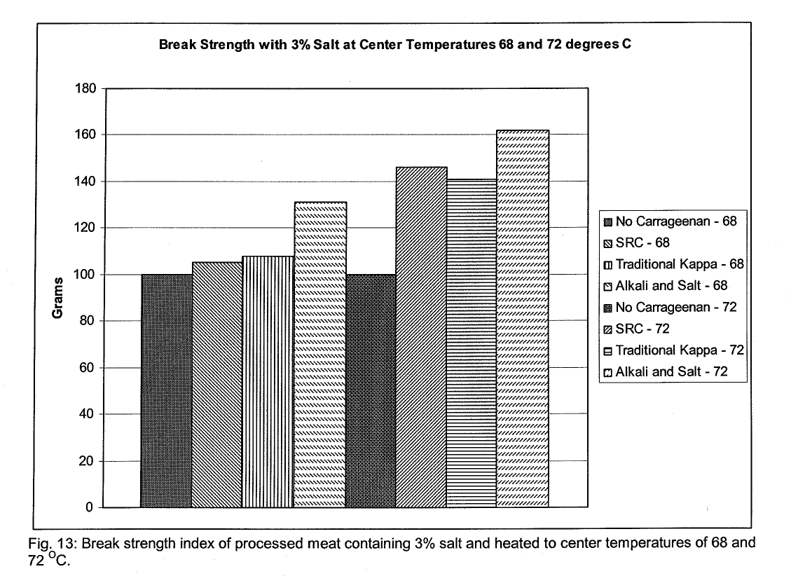

At 3% salt, FIG. 13 shows that break strength is very sensitive to processing temperature. However, the extract of the present invention provides for higher break strength at 68° C. and at 72° C. than traditional kappa carrageenan and semi refined carrageenan.

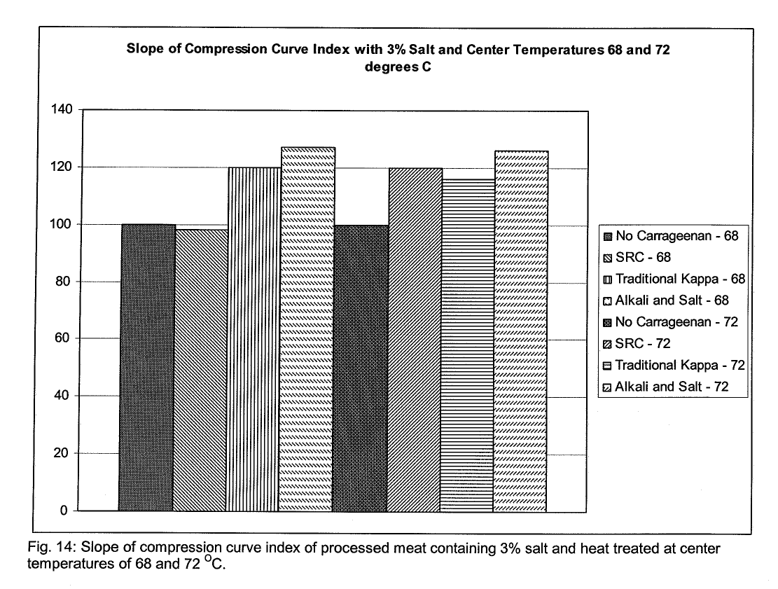

FIG. 14 shows that the slope of the compression curve is about the same for traditional kappa carrageenan and the extract of the present invention. However, the slope drops substantially for semi refined carrageenan as the center temperature during processing decreases.

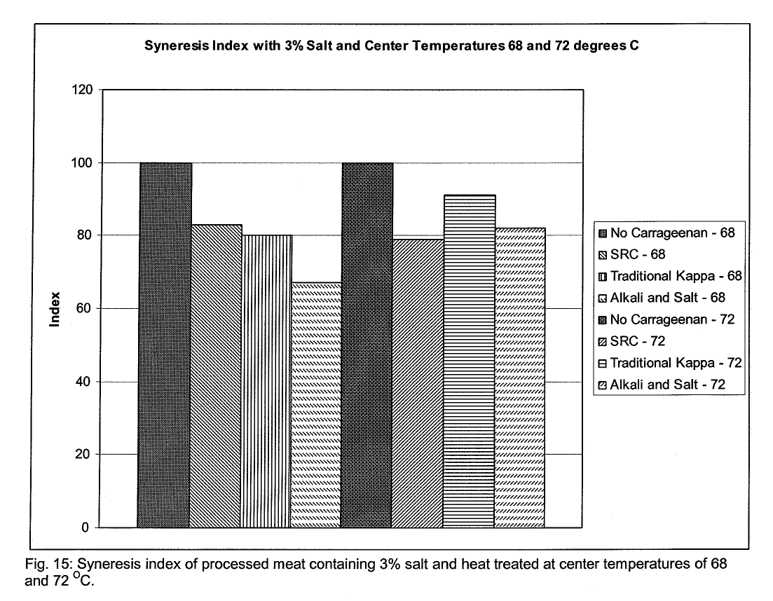

FIG. 15 shows that particularly at lower center temperatures, the extract of the present invention displays an improved water binding compared to traditional kappa carrageenan and semi refined carrageenan.

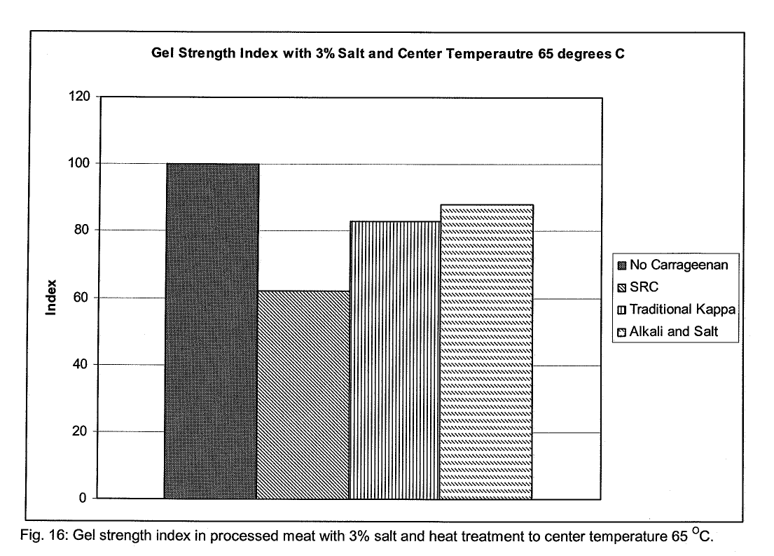

FIG. 16 shows that at 3% salt and a center temperature of 65° C., gel strengths of meats with seaweed products are all lower than the gel strength of meat without addition of seaweed product. Thus, the meat containing seaweed products is softer than the meat without. The softest product contains semi refined carrageenan, while the least soft meat contains extract of the present invention.

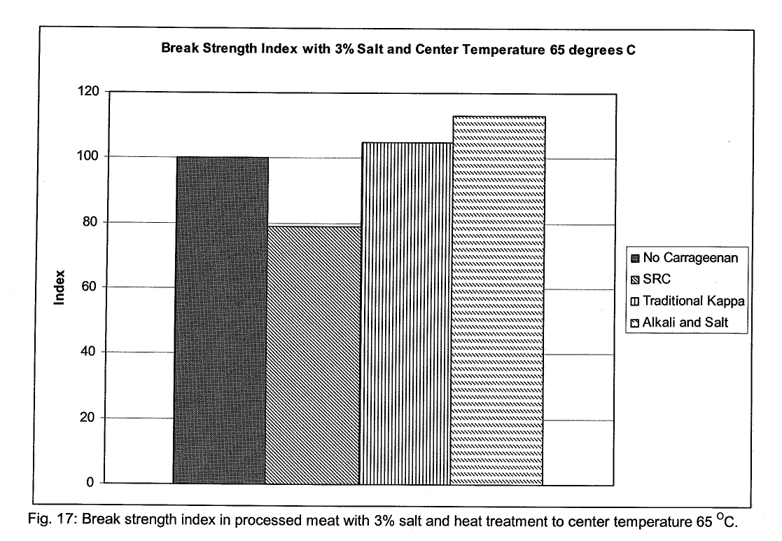

FIG. 17 shows that at 3% salt and a center temperature of 65° C., break strengths of meat containing extract of the present invention is higher than that of the other meat products.

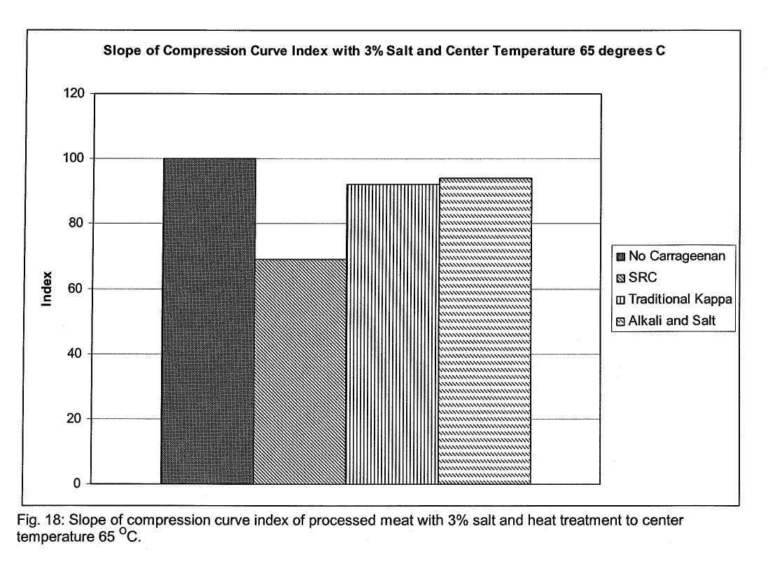

FIG. 18 shows that the slope of the compression curve is about the same for traditional kappa carrageenan and for extract of the present invention, whereas the slope in substantially lower for semi refined carrageenan.

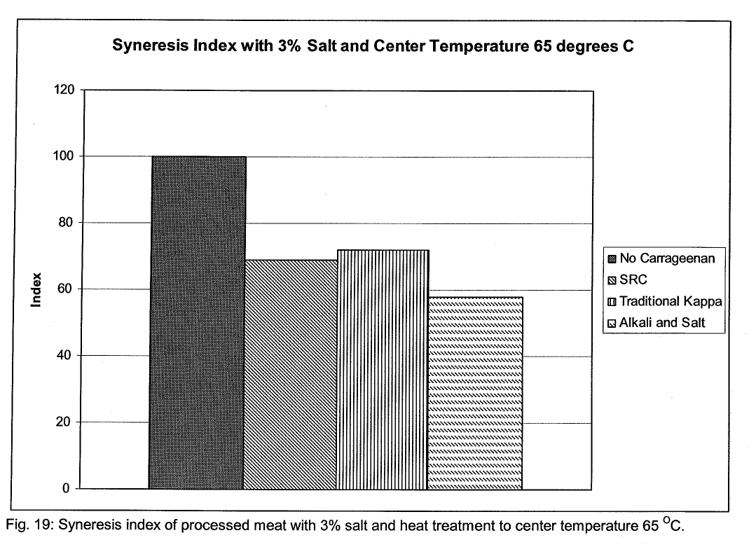

FIG. 19 shows that although the gel strength and break strength of meat products containing seaweed products are lower when applying a center temperature of 65° C. than those obtained at higher center temperatures, syneresis is still reduced, with seaweed extract of the present invention providing the biggest reduction in syneresis.

Preparation of cold-set air freshener gels. In this example, the samples “Alkali and Salt” and “Salt 10” (see above) were used to make air freshener gels without applying any heating. Different amount of dry potassium chloride was added to the crystallizing dish, and break strength and gel strength were measured at 25° C. over a period of 72 hours. The results are set forth below in Table 13.

TABLE 13

| | KCl | BS | GS | BS | GS | BS | GS |
|---|---|---|---|---|---|---|---|
| Sample | % | 24 h g | 24 h g | 48 h g | 48 h g | 72 h g | 72 h g |
| Alkali | 0.06 | 65 | 37 | 105 | 42 | 104 | 42 |
| and Salt | | | | | | | |
| Alkali | 0.12 | 120 | 57 | 200 | 68 | 189 | 71 |
| and Salt | | | | | | | |
| Alkali | 0.24 | 207 | 90 | 379 | 113 | 396 | 111 |
| and Salt | | | | | | | |
| Salt 10 | 0.06 | 122 | 52 | 143 | 56 | 193 | 75 |
| Salt 10 | 0.12 | 197 | 79 | 260 | 96 | 265 | 98 |
| Salt 10 | 0.24 | 250 | 102 | 353 | 98 | 466 | 120 |

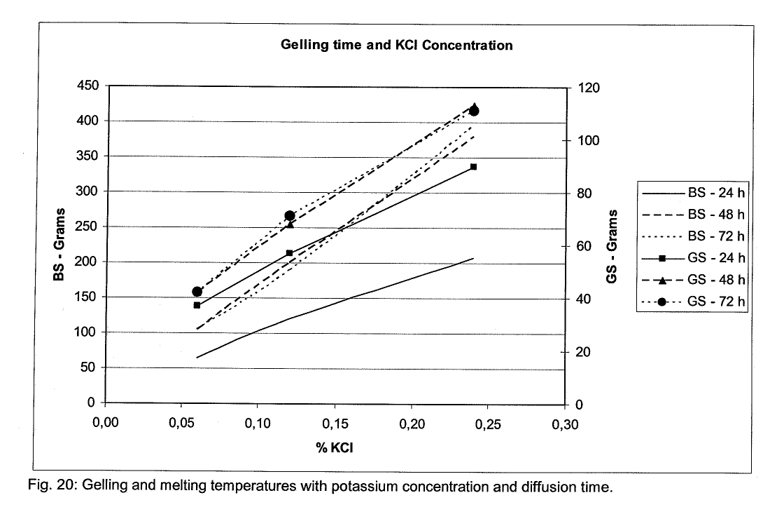

FIG. 20 shows that gelation is completed after no more than 48 hours and that gel strength and break strength increase with increasing concentration of potassium chloride.

Preparation of toothpaste. In this example, toothpastes were made with the sample “Alkali and Salt” and a traditional carrageenan products used in toothpaste, GENUVISCO carrageenan type TPC-1, manufactured by CPKelco ApS. The results are set forth in Table 14, below.

TABLE 14

| Sample | Storage, days | Storage, temp | Viscosity, cP | Index |
|---|---|---|---|---|
| TPC-1 | 3 | 25 | 205 | 100 |
| Alkali and | 3 | 25 | 489 | 239 |
| Salt | | | | |
| TPC-1 | 7 | 25 | 197 | 100 |
| Alkali and | 7 | 25 | 602 | 306 |
| Salt | | | | |
| TPC-1 | 3 | 50 | 523 | 100 |
| Alkali and | 3 | 50 | 1410 | 270 |
| Salt | | | | |
| TPC-1 | 7 | 50 | 617 | 100 |
| Alkali and | 7 | 50 | 1707 | 277 |
| Salt | | | | |

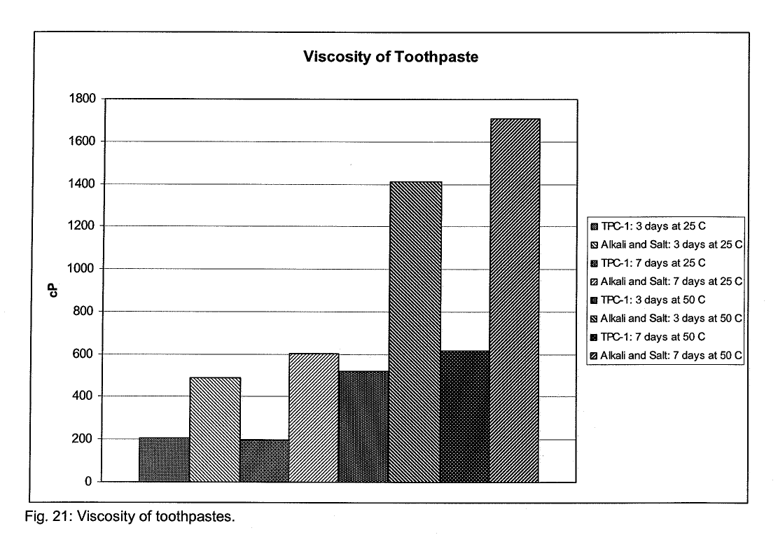

FIG. 21 shows that the carrageenan products of the present invention provide for substantially higher viscodity in toothpaste than a traditional carrageenan. In fact, the viscosity provided by carrageenan products of the present invention are more than twice as high than the viscosity provided by a traditional carrageenan for toothpaste.

It will be appreciated by those skilled in the art that changes could be made to the embodiments described above without departing from the broad inventive concept thereof. It is understood therefore that this Invention is not limited to the particular embodiments disclosed, but it is intended to cover modifications within the spirit and scope of the present invention as defined by the appended claims.

1. A carrageenan composition comprising:
sodium in the range 4.720-6.960%, preferably 5.520-6.960% and most preferably 5.770-6.960%;
potassium content of 0.015%-1.820%, preferably 0.015-0.036% and most preferably 0.015-0.025%;
calcium content of 0.032-0.210%, preferably 0.032-0.134% and most preferably 0.032-0.077%; and
magnesium content of 0.037-0.210%, preferably 0.037-0.086%, and most preferably 0.037-0.066%.
wherein the carrageenan's gelling temperatures in the range 10-20° C., preferably 10-16° C. and most preferably 10-12° C.; and the carrageenan's melting temperatures in the range 22-36° C., preferably 22-29° C. and most preferably 22-23° C.

2. A food product comprising the carrageenan according to claim 1.

3. The food product according to claim 2, wherein the food product is selected from the group comprising processed meat, poultry, or fish.

4. The food product according to claim 2, wherein the food product is an oil-in-water emulsion.

5. The food product according to claim 4, wherein the emulsion is in the form of margarine and comprises about 20 wt % to about 80 wt % fat.

6. An air freshener product comprising the carrageenan according to claim 1, wherein the air freshener gel is made in a process containing a heating step of to about 70 to about 90° C. and wherein the gel contains a non-ionic surfactant with a cloud point in the range of about 0 to about 60° C.

7. An air freshener product comprising the carrageenan according to claim 1, wherein the air freshener is a gel manufactured at room temperature through gellation by diffusion of cations, which causes gelation of the carrageenan.

8. A personal care product comprising the carrageenan according to claim 1, wherein the personal care product is in the form of a water-in-oil emulsion comprising 20-80% oil, and where said emulsion inverts at any temperature in the range 15-45° C., preferably 30-35° C. to ensure inversion on the skin surface.

9. A dentifrice product comprising the carrageenan according to claim 1.

10. A pharmaceutical product comprising the carrageenan according to claim 1, wherein the pharmaceutical product is an encapsulated drug which is released at a temperature of about 30° C. to about 40° C.

11. A carrageenan composition comprising
sodium in the range 4.390-5.730%, preferably 5.520-5.730% and most preferably 5.660-5.730%;
potassium in the range of about 0.021-1.190%, preferably 0.021-0.090% and most preferably 0.021-0.024%;
calcium in the range of 0.220-0.340%, preferably 0.220-0.270%, and most preferably 0.220-0.230%; and
magnesium in the range of 0.041-0.170%, preferably about 0.041-0.061%, and most preferably 0.041-0.055%;
wherein the carrageenan's gelling temperature is in the range 9-18° C.; and the carrageenan's melting temperatures in the range 21-34° C., preferably 21-26° C., and most preferably 21-24° C.

12. A food product comprising the carrageenan according to claim 11.

13. The food product according to claim 12, wherein the food product is selected from the group comprising processed meat, poultry, or fish.

14. The food product according to claim 12, wherein the food product is an oil-in-water emulsion.

15. The food product according to claim 14, wherein the emulsion is in the form of margarine and comprises about 20 wt % to about 80 wt % fat.

16. An air freshener product comprising the carrageenan according to claim 11, wherein the air freshener gel is made in a process containing a heating step of to about 70 to about 90° C. and wherein the gel contains a non-ionic surfactant with a cloud point in the range of about 0 to about 60° C.

17. An air freshener product comprising the carrageenan according to claim 11, wherein the air freshener is a gel manufactured at room temperature through gellation by diffusion of cations, which cause gelation of the carrageenan.

18. A personal care product comprising the carrageenan according to claim 11, wherein the personal care product is in the form of a water-in-oil emulsion comprising 20-80% oil, and where said emulsion inverts at any temperature in the range 15-45° C., preferably 30-35° C. to ensure inversion on the skin surface.

19. A dentifrice product comprising the carrageenan according to claim 11.

20. A pharmaceutical product comprising the carrageenan according to claim 1, wherein the pharmaceutical product is an encapsulated drug which is released at a temperature of about 30° C. to about 40° C.

21. A carrageenan composition comprising:
sodium in the range of 4.870-6.910%, preferably 5.770-6.910% and more preferably 6.010-6.910%;
potassium in the range of 0.014-1.180%, preferably 0.014-0.068% and more preferably 0.0.14-0.035%;
calcium in the range of 0.073-0.260%, preferably 0.073-0.200% and most preferably 0.073-0.146%; and
magnesium in the range of 0.010-0.290%, preferably 0.010-0.160% and more preferably 0.010-0.103%.
wherein the carrageenan's gelling temperature is in the range 9-19° C., preferably 9-15° C. and most preferably 9-13° C.; and the carrageenan's melting temperatures in the range 21-35° C., preferably 21-29° C. and most preferably 21-26° C.

22. A food product comprising the carrageenan according to claim 21.

23. The food product according to claim 22, wherein the food product is selected from the group comprising processed meat, poultry, or fish.

24. The food product according to claim 22, wherein the food product is an oil-in-water emulsion.

25. The food product according to claim 24, wherein the emulsion is in the form of margarine and comprises about 20 wt % to about 80 wt % fat.

26. An air freshener product comprising the carrageenan according to claim 21, wherein the air freshener gel is made in a process containing a heating step of to about 70 to about 90° C. and wherein the gel contains a non-ionic surfactant with a cloud point in the range of about 0 to about 60° C.

27. An air freshener product comprising the carrageenan according to claim 21, wherein the air freshener is a gel manufactured at room temperature through gellation by diffusion of cations, which cause gelation of the carrageenan.

28. A personal care product comprising the carrageenan according to claim 21, wherein the personal care product is in the form of a water-in-oil emulsion comprising 20-80% oil, and where said emulsion inverts at any temperature in the range 15-45° C., preferably 30-35° C. to ensure inversion on the skin surface.

29. A dentifrice product comprising the carrageenan according to claim 21.

30. A pharmaceutical product comprising the carrageenan according to claim 21, wherein the pharmaceutical product is an encapsulated drug which is released at a temperature of about 30° C. to about 40° C.

31. A carrageenan composition comprising:
sodium in the range 4.870-6.910%, preferably 5.770-6.910% and more preferably 6.010-6.910%;
potassium in the range of 0.014-1.180%, preferably 0.014-0.068% and more preferably 0.014-0.035%;
calcium in the range of 0.073-0.260%, preferably 0.073-0.200% and most preferably 0.073-0.146%; and
magnesium in the range of 0.010-0.290%, preferably 0.010-0.160% and more preferably 0.010-0.103%;
wherein the carrageenan's gelling temperatures in the range 9-19° C., preferably 9-15° C. and most preferably 9-13° C.; and the carrageenan's melting temperature is in the range 21-35° C., preferably 21-29° C. and most preferably 21-26 ° C.

32. A food product comprising the carrageenan according to claim 31.

33. The food product according to claim 32, wherein the food product is selected from the group comprising processed meat, poultry, or fish.

34. The food product according to claim 32, wherein the food product is an oil-in-water emulsion.

35. The food product according to claim 34, wherein the emulsion is in the form of margarine and comprises about 20 wt % to about 80 wt % fat.

36. An air freshener product comprising the carrageenan according to claim 31, wherein the air freshener gel is made in a process containing a heating step of to about 70 to about 90° C. and wherein the gel contains a non-ionic surfactant with a cloud point in the range of about 0 to about 60° C.

37. An air freshener product comprising the carrageenan according to claim 31, wherein the air freshener is a gel manufactured at room temperature through gellation by diffusion of cations, which cause gelation of the carrageenan.

38. A personal care product comprising the carrageenan according to claim 31, wherein the personal care product is in the form of a water-in-oil emulsion comprising 20-80% oil, and where said emulsion inverts at any temperature in the range 15-45° C., preferably 30-35° C. to ensure inversion on the skin surface.

39. A dentifrice product comprising the carrageenan according to claim 31.

40. A pharmaceutical product comprising the carrageenan according to claim 31, wherein the pharmaceutical product is an encapsulated drug which is released at a temperature of about 30° C. to about 40° C.

41. The carrageenan composition according to claims 1, 11, 21, or 31, which has the following gelling and melting temperatures when incorporated into an air freshener gel:

| | | T_{G} | T_{M} |
|---|---|---|---|
| System | Carrageenan % | ° C. | ° C. |
| Air gel | 0.50 | 3-11 | 13-31 |
| | 1.00 | 10-19 | 23-35 |
| | 1.50 | 13-22 | 28-38 |

42. The carrageenan composition according to claims 1, 11, 21, or 31, which has the following gelling and melting temperatures when incorporated into demineralized water:

| | | T_{G} | T_{M} |
|---|---|---|---|
| System | Carrageenan % | ° C. | ° C. |
| Demineralised | 1.00 | 0-5 | 0-5 |
| water | 2.00 | 0-10 | 0-18 |
| | 3.00 | 0-15 | 0-29 |

43. The carrageenan composition according to claims 1, 11, 21, or 31, which has the following gelling and melting temperatures when incorporated into demineralized water and sodium chloride:

| | | | T_{G} | T_{M} |
|---|---|---|---|---|
| System | Carrageenan % | NaCl % | ° C. | ° C. |
| Demineralised | 0.60 | 1.00 | 2-9 | 9-19 |
| water and NaCl | 0.60 | 3.00 | 18-24 | 24-27 |
| | 0.60 | 5.00 | 29-35 | 34-39 |

44. The carrageenan composition according to claims 1, 11, 21, or 31, which has the following gelling and melting temperatures when incorporated into demineralized water containing potassium chloride:

| | | T_{G} | T_{M} |
|---|---|---|---|
| Carrageenan % | KCl % | ° C. | ° C. |
| 1.00 | 0.00 | 0-5 | 0-7 |
| | 0.05 | 3-10 | 13-27 |
| | 0.20 | 27-29 | 45-49 |
| | 0.30 | 36-43 | 51-60 |

45. The carrageenan composition according to claims 1, 11, 21, or 31, which has the following gelling and melting temperatures when incorporated into demineralized water containing calcium chloride:

| Carrageenan % | CaCl₂•2H₂O | T_{G} | T_{M} |
|---|---|---|---|
| 1.00 | 0.08 | 15-34 | 30-44 |
| | 0.16 | 42-55 | 52-61 |
| | 0.32 | 60-74 | 67-83 |
